(19) United States
(12) Patent Application Publication
(10) Pub. No.: US 2006/0128941 A1
(43) Pub. Date: Jun. 15, 2006

(54) Pokeweed antiviral protein polypeptides with antiviral activity

(76) Inventors: Faith M. Uckun, White Bear Lake, MN (US); Francis Rajamohan, Groton, CT (US)

Correspondence Address: MERCHANT & GOULD PC, P.O. BOX 2903, MINNEAPOLIS, MN, 55402-0903, US

(21) Appl. No.: 10/518,142

(22) PCT Filed: Jun. 17, 2003
(86) PCT No.: PCT/US03/19141

§ 371 Date: Jan. 3, 2006

Related U.S. Application Data
(60) Provisional application No. 60/389,649, filed on Jun. 17, 2002.

(51) Int. Cl. A61K 31/70; C07K 14/00; A01N 43/04; C07K 16/00; C07K 7/00; C07K 5/00; C07K 4/00; C07K 2/00; C07K 17/00; A61K 38/00

(52) U.S. Cl. 530/300; 514/44

(57) ABSTRACT

A molecular model of pokeweed antiviral protein (PAP)-RNA interactions was used to rationally engineer FLP-102 (¹⁵¹AA¹⁵²) and FLP-105 (¹⁹¹AG¹⁹²) as nontoxic PAP proteins with potent anti-HIV activity. FLP-102 and FLP-105 have been produced in E. coli and tested both in vitro as well as in vivo. These proteins depurinate HIV-1 RNA much better than ribosomal RNA and are more potent anti-HIV agents than native PAP or recombinant wild-type PAP. They are substantially less toxic than native PAP in BALB/c mice and exhibit potent in vivo activity against genotypically and phenotypically NRTI-resistant HIV-1 in a surrogate Hu-PBL-SLID mouse model of human AIDS. Rationally engineered nontoxic recombinant PAP proteins such as FLP-102 and FLP-105 may provide the basis for effective salvage therapies for patients harboring highly drug resistant strains of HIV-1. The documented in vitro potency of FLP-102 and FLP-105, their in vivo antiretroviral activity in HIV-infected Hu-PBL SCID mice, and their favorable toxicity profile in BALB/c mice warrant the further development of these promising new biotherapeutic agents.

This application is being filed as a PCT International Patent Application in the name of Parker Hughes Institute, a U.S. national corporation and resident, (Applicant for all countries except US) and Fatih M. Uckun, a U.S. citizen and resident (Applicant for US only), on 17 Jun. 2003, designating all countries and claiming priority to U.S. Ser. No. 60/389,649 filed on 17 Jun. 2002.

### BACKGROUND

Combination antiretroviral therapy has become the standard of care for patients with HIV infection in the United States (Gottlieb M S, 2001, N Engl J Med, 344(23): 1788-91; Sepkowitz K A, 2001, N Engl J Med, 344(23):1764-72; Freedberg K A, et al., 2001, N Engl J Med 344(11):824-31; Richman D D, 2001, HIV chemotherapy, Nature, 410:995-1001; Shafer R W and Vuitton D A, 1999, Biomed Pharmacother, 53(2):73-86; Starr S E, et al.,1999, Pediatric AIDS Clinical Trials Group 382 Team. N Engl J Med 341(25):1874-81; Rey D, et al., 2001, J Acquir Immune Defic Syndr, 27(5):459-62). Anti-retroviral treatment regimens employing combinations of drugs from at least two of the three classes of antiretroviral therapy, namely nucleoside analog RT inhibitors (NRTI), non-nucleoside analog RT inhibitors (NNRTI), and protease inhibitors (PI), exhibit a potent and sustained antiviral effect and confer consistent long-term viral suppression in patients with HIV infection (Gottlieb M S, 2001, N Engl J Med, 344(23):1788-91; Sepkowitz K A, 2001, N Engl J Med, 344(23):1764-72; Freedberg K A, et al., 2001, N Engl J Med 344(11):824-31; Richman D D, 2001, HIV chemotherapy, Nature, 410:995-1001; Shafer R W and Vuitton D A, 1999, Biomed Pharmacother, 53(2):73-86; Starr S E, et al., 1999, Pediatric AIDS Clinical Trials Group 382 Team. N Engl J Med 341(25):1874-81; Rey D, et al., 2001, J Acquir Immune Defic Syndr, 27(5):459-62). The individual components of these combination regimens cannot select for drug-resistant viruses. However, the emergence of antiviral drug resistance limits their clinical benefit (Ross L, et al., 2001, AIDS Res Hum Retroviruses, 17(12): 1107-15; Picard V, et al., 2001, J Infect Dis, 184(6):781-4; Suzuki K, et al., AIDS Res Hum Retroviruses, 17(13):1293-6; Izopet J, et al., 1999, J Med Virol, 59(4):507-11; Venturi G, et al., 1999, Eur J Clin Microbiol Infect Dis, 18(4):274-82; Kuritzkes D R, et al., 2000, J Infect Dis, 181(2):491-7; O'Brien W A, 2000, Clin Infect Dis, 30 Suppl 2:S185-92; Pillay D, et al., 2000, Rev Med Virol, 10(4):231-53; Briones C, et al., 2000, AIDS, 14(11): 1659-60). This resistance is a consequence of the high mutation rate and fast replication of HIV and the selective effect of these drugs, which favors emergence of mutations that can establish clinical drug resistance. Recent results indicate that failure of the highly active antiretroviral therapy (HAART), which typically includes at least 2 NRTI and a protease inhibitor or a NNRTI, results from the multiplicity of mutations that confer genotypic resistance to almost all available antiretroviral drugs (Ross L, et al., 2001, AIDS Res Hum Retroviruses, 17(12):1107-15; Picard V, et al., 2001, J Infect Dis, 184(6):781-4; Suzuki K, et al., AIDS Res Hum Retroviruses, 17(13):1293-6; Izopet J, et al., 1999, J Med Virol, 59(4):507-1 1; Venturi G, et al., 1999, Eur J Clin Microbiol Infect Dis, 18(4):274-82; Kuritzkes D R, et al., 2000, J Infect Dis, 181(2):491-7; O'Brien W A, 2000, Clin Infect Dis, 30 Suppl 2:S 185-92; Pillay D, et al., 2000, Rev Med Virol, 10(4):231-53; Briones C, et al., 2000, AIDS, 14(11):1659-60). In these patients, genotypic resistance tests confirm the lack of alternative salvage therapy strategies based on the currently available antiretroviral drugs (Ross L, et al., 2001, AIDS Res Hum Retroviruses, 17(12):1107-15; Picard V, et al., 2001, J Infect Dis, 184(6):781-4; Suzuki K, et al., AIDS Res Hum Retroviruses, 17(13):1293-6; Izopet J, et al., 1999, J Med Virol, 59(4):507-1 1; Venturi G, et al., 1999, Eur J Clin Microbiol Infect Dis, 18(4):274-82; Kuritzkes D R, et al., 2000, J Infect Dis, 181(2):491-7; O'Brien W A, 2000, Clin Infect Dis, 30 Suppl 2:S 185-92; Pillay D, et al., 2000, Rev Med Virol, 10(4):231-53; Briones C, et al., 2000, AIDS, 14(11):1659-60). Patients failing on HAART constitute a reservoir of multi-drug resistant HIV that may limit treatment options in the future. The frequency of genotypic and phenotypic drug-resistant HIV is increasing among therapy-naive HIV-infected sero-converters (Ross L, et al., 2001, AIDS Res Hum Retroviruses, 17(12):1107-15; Picard V, et al., 2001, J Infect Dis, 184(6):781-4; Suzuki K, et al., AIDS Res Hum Retroviruses, 17(13):1293-6; Izopet J, et al., 1999, J Med Virol, 59(4):507-1 1; Venturi G, et al., 1999, Eur J Clin Microbiol Infect Dis, 18(4):274-82; Kuritzkes D R, et al., 2000, J Infect Dis, 181(2):491-7; O'Brien W A, 2000, Clin Infect Dis, 30 Suppl 2:S185-92;

Pillay D, et al., 2000, Rev Med Virol, 10(4):231-53; Briones C, et al., 2000, AIDS, 14(11):1659-60). Thus, the transmission of drug-resistant HIV is a serious problem that merits further attention by public health officials as well as virologists and clinicians. Therefore, there is an urgent need for new anti-HIV agents capable of inhibiting the replication of drug-resistant HIV.

Pokeweed antiviral protein (PAP) is one agent that has shown promise for use against drug resistant HIV. PAP is a 29-kDa naturally occurring antiviral agent that can be isolated from the leaves of the pokeweed plant, Phytolacca americana (Irvin J D and Uckun, 1992, Pharmacol Ther, 55:279-302; Zarling J M, et al., 1990, Nature, 347:92-95). PAP has a unique ability to depurinate HIV-1 RNA (Rajamohan F, et al., 1999, Biochemical and Biophysical Research Communications, 263(2): 419-424; Rajamohan F, et al., 1999, BBRC, 260(2): 453-458). PAP exhibits potent antiviral activity against NRTI-resistant primary clinical HIV-1 isolates (Erice A, et al., 1993, Antimicrob Agents Chemother, 37:835-838). Both zidovudine (ZDV)-sensitive and ZDV-resistant clinical HIV-1 isolates were found to be more sensitive to PAP than to ZDV, by more than 4-logs (Erice A, et al., 1993, Antimicrob Agents Chemother, 37:835-838). The gene for PAP has been cloned and procedures have been established for large-scale production and purification of the cloned recombinant PAP (Rajamohan F, et al., 1999, Protein Expression and Purification, 16(2): 359-368, 1999; Rajamohan F, et al., 2000, Protein Expression and Purification, 18(2):193-201). Recombinant PAP has been tested against a broad panel of viruses in vitro and documented that it is as active as native PAP against both DNA and RNA viruses (Rajamohan F, et al., 1999, Protein Expression and Purification, 16(2): 359-368, 1999; Rajamohan F, et al., 2000, Protein Expression and Purification, 18(2):193-201). The X-ray crystal structure of PAP has also been determined at a resolution of 2.1-Å (Kurinov I, et al., 1999, Protein Science, 8(11): 2399-2405; Kurinov I V, et al., 1999, Protein Science, 8(9): 1765-1772).

More recently, a molecular model of PAP-HIV RNA interactions (Rajamohan F, et al., 2000, Journal of Biological Chemistry, 275(5):3382-3390; Rajamohan, F., et al., 2001, Journal of Biological Chemistry, 276(26): 24075-81; Rajamohan, F., et al., 2001, Biochemistry, 40:9104-14, 2001) and has been used for the rational design of PAP mutants with potent anti-HIV activity. Rational design of PAP mutants with potent anti-HIV activity serves as a promising tool for developing new and useful anti-HIV agents. However, not much is known about which areas of the PAP molecule, other than the active site, can be modified to produce PAP mutants with anti-HIV activity.

### SUMMARY OF THE INVENTION

The present application provides modified PAP polypeptides having one or more modifications, relative to wild-type PAP, within one or more regions other than the active site such that the activity of the modified PAP towards viral RNA is increased relative to wild-type PAP. The viral RNA can be retroviral RNA. Such retroviruses include HIV-1 and drug-resistant HIV-1. In one embodiment, the modified PAP also exhibits decreased activity towards ribosomal RNA relative to wild-type PAP.

In one embodiment of the invention, a PAP polypeptide includes a modification, relative to wild-type PAP in a region other than the active site and alters the relative confirmation of at least one amino acid, which is at least partially buried in wild-type PAP. The region other than the active site can be a hydrophobic region. In one embodiment, the modification is in a region more than 15 Å away from the active site.

In one embodiment, the modification of a PAP polypeptide occurs within the α helix 4-loop-α helix 5 region. The modification can be localized to a hydrophobic sub-region where at least one amino acid of wild-type PAP is buried. For example, the modification of PAP in this region can occur at one or more of the following amino acids: 76, 151, 152, 158, 162, or 166, referring to the numbering of amino acids in wild-type PAP. For example, the amino acid at position 151 can be mutated from lysine to alanine or the amino acid at position 152 can be modified from isoleucine to alanine.

In one embodiment, the modification of a PAP polypeptide occurs within the C-terminal region of α helix 6. The modification of PAP in this region can occur at, for example, one or more of the following amino acids: 13, 16, 142, 188, 191, or 192, referring to the numbering of amino acids in wild-type PAP. For example, the amino acid at position 191 can be mutated from phenylalanine to alanine or the amino acid at position 192 can be modified from asparagine to glycine.

The invention also provides compositions comprising one or more modified PAP polypeptide according to the invention. Such compositions can be pharmaceutical compositions.

The invention also provides methods for inhibiting viral replication comprising contacting a viral RNA with one or more modified PAP polypeptide according to the invention.

The invention also provides methods for depurinate viral RNA comprising contacting the viral RNA with one or more modified PAP polypeptide according to the invention.

The invention also provides methods for treating viral infections including administering one or more modified PAP polypeptide according to the invention to a subject in need thereof. In one embodiment, the method for treating viral infections further includes the addition of one or more antiviral agents. The antiviral agents can be nucleoside analogs inhibitors of reverse transcriptase, non-nucleoside inhibitors of reverse transcriptase, viral protease inhibitors, and the like.

### BRIEF DESCRIPTION OF THE DRAWINGS

FIG. 1 is a representation of ribbon and space-filling models of PAP (gray ribbons) complexed with ribosomal RNA (left, in white) and viral RNA (right, in white) molecules.

FIG. 2 shows the analysis of wild-type and mutant recombinant PAP proteins by (A) Coomassie blue-stained SDS-12% polyacrylamide gel electrophoresis and (B) Western blot.

FIG. 3 shows the effect on protein synthesis of wild-type and mutant PAP protein in an in vitro rabbit reticulocyte lysate translation system (i.e. via inhibition of ribosome binding). (A) FLP-102 and single residue mutants FLP-100 and FLP-101. (B) FLP-105 and single residue mutants FLP-103 and FLP-104.

FIG. 4 shows the in vitro depurination of E. coli ribosomal RNA by wild-type and mutant PAP proteins, as measured by aniline cleavage and fragment separation on 6% urea/polyacrylamide gels.

FIG. 5 shows the association of wild-type and mutant PAP proteins with either native ribosomes or in vitro synthesized ribosomal protein L3. (A) Association of mutant PAP proteins with ribosomes isolated from rabbit reticulocyte-enriched blood. (B) Association of wild-type and mutant PAP proteins with in vitro synthesized ribosomal protein L3.

FIG. 6 shows the depurination of E. coli and HIV-1 RNA by wild-type and mutant PAP proteins. Inset: Standard curve of adenine and guanine standards.

FIG. 7 shows the in vivo toxicity of mutant PAP proteins, FLP-102 and FLP-105, in BALB/c mice.

### DETAILED DESCRIPTION OF THE INVENTION

Abbreviations

(FLP) Full-length pokeweed antiviral protein (PAP)
(HAART) Highly active anti-retroviral therapy
(HCMV) Human cytomegalovirus
(NRTI) Nucleoside reverse transcriptase inhibitor
(NNRTI) Non-nucleoside reverse transcriptase inhibitor
(PI) Protease inhibitor
(PAP) Pokeweed antiviral protein
(PBMC) Peripheral blood mononuclear cells
(PBS-PAP) Plasmid (Bluescript) containing the full-length PAP insert
(SDS-PAGE) Sodium dodecyl sulfate-polyacrylamide gel electrophoresis
(WT) Wild-type
   Definitions

As used herein, “wild-type PAP” means the PAP amino acid sequence essentially as depicted in Table 2. The wild-type PAP amino acid sequence of Table 2, of which amino acids 1-262 correspond to SEQ ID NO: 1, contains a 22-amino acid N-terminal signal peptide and a 29 amino acid C-terminal extension (amino acids enumerated 263-291; illustrated in Table 2). Upon expression in eukaryotic cells, the N-terminal 22-amino acid sequence is typically co-translationally cleaved, yielding a polypeptide having a molecular weight of about 32 kDa, which is then further processed by the cleavage of the C-terminal 29 amino acids, yielding mature, wild-type PAP (hereinafter “PAP (1-262)”) (i.e., that which is isolated from Phytolacca americana leaves), having a molecular weight of about 29 kDa Irvin et al., Supra, Pharmacology and Therapeutics 55:279-302 (1992). Thus, by “wild-type, mature PAP”, it is meant that the amino acid sequence corresponds essentially to the PAP amino acid sequence 1-262 shown in Table 2. “Wild-type PAP” as used herein can refer to wild-type PAP or wild-type mature PAP. Reference to amino acids at various positions of PAP or modified PAP polypeptides herein is reference to the corresponding amino acid position in wild-type PAP.

As used herein, “modified PAP” means a PAP polypeptide, wherein one or more amino acids are altered, relative to wild-type PAP, in one or more regions other than the active site such that the activity of the modified PAP towards viral RNA is increased relative to wild-type PAP. The alterations to amino acids include point mutations, deletion mutations, insertion mutations, chemical modification, and the like.

As used herein “PAP activity” means the ability of PAP to either depurinate RNA or inhibit viral replication. The increased activity of modified PAP referred to herein means that the rate or amount of catalyzed depurination or inhibition of viral replication is greater for modified PAP than for wild-type PAP. With regard to ribosomal RNA, the activity of PAP refers to the removal of a single adenine from the highly conserved, surface-exposed “α-sarcin/ricin” loop located near the 3′ terminus of the large rRNA of prokaryotic and eukaryotic ribosomes. Irvin et al., Pharmacology and Therapeutics 55:279-302 (1992).

As used herein, “active site” of PAP means the cavity like surface of PAP containing and within about 10 Å of the catalytic residues of PAP. The catalytic residues of PAP are the residues that catalyze the depurination of RNA. A region of PAP other than the active site includes those regions more than about 10 Å away from the catalytic residues of PAP. The regions other than the active site can about 10-15 Å away from the catalytic residues of PAP, about 15-20 Å away from the catalytic residues of PAP, more than about 20 Å away from the catalytic residues of PAP, or more than about 25 Å away from the catalytic residues of PAP. The catalytic residue R179 can be used as a point of reference to determine distance of an amino acid or region from the active site.

1. Modeling of PAP Interactions With RNA

One method for determining whether modified PAP polypeptides exhibit increased activity towards viral RNA is to model the interactions of PAP with viral RNA as indicated herein or as known in the art. The modified PAP polypeptides tested in silico can then optionally be made in vitro as described herein or according to any known technique and then can be further optionally tested as described herein or according to any known technique.

2. Modification of PAP

Modified PAP polypeptides of the invention can be modified by substituting, at a given position, one amino acid residue for another. This can be accomplished by, e.g., introducing point mutations into a nucleic acid encoding for the modified PAP. Techniques for achieving such modified PAP molecules are described herein and are know in the art.

The rationale for determining which amino acids to substitute for one another can be based on physical size, polarity, and other well-known properties of amino acids as known in the art. For example, if a PAP polypeptide of the invention contains an alanine substitution at a particular position, it is more likely that other non-polar amino acids, as opposed to polar amino acids, will serve as suitable substitutions at that position. Also by way of example, if a PAP polypeptide of the invention contains an alanine substitution at a particular position, it is more likely that similarly sized amino acids, as opposed to dissimilarly sized amino acids, will serve as suitable substitutions at that position. Some of the relevant properties of amino acids are listed below in Table 1. The properties in Table I can be helpful for determining which amino acid substitutions will be suitable for the modified PAP polypeptides of the invention.

TABLE 1
Properties of amino acids.

| | | | Residue | |
|---|---|---|---|---|
| | | Molecular | volume (Å) | Side Chain |
| Amino Acid | Symbol | Weight | (Surface Area) | pKa |
| Alanine | A, Ala | 71.079 | 88.6 (115) | — |
| Arginine | R, Arg | 156.188 | 173.4 (225) | ˜12 |
| Asparagine | D, Asn | 114.104 | 111.1 (150) | 4.5 |
| Aspartic Acid | D, Asp | 115.089 | 114.1 (160) | — |
| Cysteine | C, Cys | 103.145 | 108.5 (135) | 9.1-9.5 |
| Glutamine | Q, Gln | 128.131 | 138.4 (190) | 4.6 |
| Glutamic Acid | E, Glu | 129.116 | 143.8 (180) | — |
| Glycine | G, Gly | 57.052 | 60.1 (75) | — |
| Histidine | H, His | 137.141 | 153.2 (195) | 6.2 |
| Isoleucine | I, Ile | 113.160 | 166.7 (175) | — |
| Leucine | L, Leu | 113.160 | 166.7 (170) | — |
| Lysine | K, Lys | 128.17 | 168.6 (200) | 10.4 |
| Methionine | M, Met | 131.199 | 162.9 (185) | — |
| Phenylalanine | F, Phe | 147.177 | 189.9 (210) | — |
| Proline | P, Pro | 97.117 | 112.7 (145) | — |
| Serine | S, Ser | 87.078 | 89.0 (115) | — |
| Threonine | T, Thr | 101.105 | 116.1 (140) | — |
| Tryptophan | W, Trp | 186.213 | 227.8 (255) | — |
| Tyrosine | Y, Tyr | 163.176 | 193.6 (230) | 9.7 |
| Valine | V, Val | 99.133 | 140.0 (155) | — |

Small polar amino acids include: P, D, E, N, and Q. Small non-polar amino acids include: G, A, S, T, and C. Large polar amino acids include: K, R, H, W, and Y. Large non-polar amino acids include: V, I, M, F, and L.

In addition, the following groups of amino acids have been taught in the art as producing conservative results for amino acid residues that are solvent exposed, and thus can be expected to produce similar results: 1) G, A, S, T; 2) N, D, E, Q; 3) M, I, V, L; 4) K, R; and 5) F, Y, W. (PROWL—Amino Acid Property website at http://prowl.rockefeller.edu/aainfo/contents.htm).

For interior residues, the following groups of amino acids have been taught in the art as producing conservative results: 1) G, A, S, T; 2) N, D, E; 3) M, L, I, V; 4) F, Y, W; 5) H, R; 6) V,A; and 7) L, F. (PROWL—Amino Acid Property website at http://prowl.rockefeller.edu/aainfo/contents.htm).

Any suitable amino acid substitution can produce a modified PAP polypeptide of the invention.

In one embodiment, a modified PAP polypeptide comprises an amino acid modification in the a helix 4-loop-α helix 5 region. An exemplary modification is presented below in the Examples, where a K151A, a I152A, or both a K151A and an I152A substitution produced a modified PAP of the invention. In wild-type PAP I152a is mostly buried within a hydrophobic region of the α helix 4-loop-α helix 5. A substitution of alanine for isoleucine produced a modified PAP polypeptide of the invention. Thus substitutions of additional small non-polar amino acid for the large non-polar amino acid at this position can also be modified PAP polypeptides of the invention. In addition, similar modifications of other amino acids within this region would be expected to produce modified PAP polypeptides according to the invention. Other amino acids in this region include F158, T162, T166, and Y76. Substitution of smaller amino acids for one or more of these amino acids would be expected to produce a modified PAP polypeptide according to the invention. Additionally, other modifications that would result in a similar conformational change in the α helix 4-loop-α helix 5 region would be expected to produce a modified PAP polypeptide according to the invention. Just a F158, T162, T166, and Y76 are in the vicinity of I152 with regard to tertiary structure, amino acid K151 neighbors I152 in primary structure. A K151A mutation resulted in a modified PAP polypeptide according to the invention. Thus, just as modifying amino acids within the hydrophobic region of α helix 4-loop-α helix 5 can result in a modified PAP polypeptide of the invention, modification of neighboring amino acids with regard to primary structure can result in a modified PAP polypeptide of the invention. Amino acids neighboring in primary structure include: 151, 153, 157, 159, 161, 163, 165, 167, 75, and 77.

In one embodiment, a modified PAP polypeptide comprises an amino acid modification in the C-terminal region of α helix 6. An exemplary modification is presented below in the Examples, where a F191A, a N192G, or both a F191A and a N192A substitution produced a modified PAP of the invention. In wild-type PAP, F191 a is mostly buried within the C-terminal region of α helix 6 and is in hydrophobic contact with the side chains of I142, I13, Y16, and the hydrophobic portion of K188. Thus, similar modifications of I142, I13, Y16, and K188, or modifications producing a similar change in conformation in this region, would be expected to produce a modified PAP polypeptide of the invention. Similarly, modification of neighboring amino acids in primary structure would also be expected to produce modified PAP polypeptides of the invention, as shown with N192G. Thus, modification of one or more of the amino acids at the following positions in or around the C-terminal region of a helix 6 can produce modified PAP polypeptides according to the invention: 190, 191, 192, 141, 142, 143, 12, 13, 14, 15, 16, 16, 187, 188, and 189.

In addition to amino acid substitution, amino acids in modified PAP polypeptides can be modified through other known means in the art, such as chemical modification, to produce a desired effect.

3. Conjugation

Modified PAP polypeptides of the invention can be conjugated to a targeting moiety to further enhance their ability to inhibit viral replication or to catalyze viral RNA depurination. One method for targeting the modified PAP polypeptides of the invention to targeting moieties is immunoconjugation. Conjugation of PAP to a suitable antibody can be achieved through methods known in the art.

The antiviral activity of PAP can be greatly enhanced and made highly cell selective by conjugation to antibodies specific for cell-surface receptors. One such immunoconjugate containing PAP has been developed and tested by our group against a member of the herpes family, human cytomegalovirus (HCMV). In this study, the antiviral action of PAP was found to be enhanced by chemically coupling it to an antibody. Gehrz et al., “Treatment of human cytomegalovirus (HCMV) with novel antiviral immunoconjugates”, in Progress in Cytomegalovirus Research, Landin, M. P. Ed., Elsevier Science Publishers BV, Amsterdam, p. 353 (1991). PAP-antibody conjugates were prepared with monoclonal antibodies specific for the low-density lipoprotein receptor (LDLr) and the HCMV envelope glycoprotein gp55, which is expressed on HCMV infected cells, and tested for antiviral effects. The conjugate prepared with PAP and anti-LDLr increased the antiviral action of PAP 1000-fold, resulting in 50% reduction in plaque formation at 1 ng/mL. Conjugation of PAP to anti-gp55 did not increase the antiviral activity observed for PAP alone. Gehrz et al., cited supra. These studies show that the antiviral activity of PAP can be significantly increased by conjugation to cell surface directed antibodies, but that the antibodies must be targeted to cell surface proteins that are capable of being internalized.

a. Immunoconjugation

Several requirements should be fulfilled for an immunotoxin to be most effective. Pastan et al., Cell, 47, 641 (1986). First of all, the immunoconjugates should be specific and should not react with uninfected tissues. Binding to tissues that do not express antigen can be reduced by removal of the nonspecific natural cell-binding subunits or domains of the toxin. Furthermore, because plant glycoprotein toxins contain mannose oligosaccharides that bind to cells of the reticuloendothelial system and, in some cases, also contain fucose residues that are recognized by the receptors on hepatocytes, deglycosylation of plant toxins may be required to avoid rapid clearance and potential cytotoxic effects on these cells. Secondly, the linkage of the toxin to the antibody should not impair the capacity of the antibody to bind to the antigen. Third, the immunotoxin must be internalized into the endosomic vesicles. Thus, toxins directed by monoclonal antibodies to surface receptors that are normally internalized may be more active than those directed toward noninternalizing cell surface molecules. Fourth, the active component of the toxin must translocate into the cytoplasm. Finally, for in vivo therapy, the linkage must be sufficiently stable to remain intact while the immunotoxin passes through the tissues of the patient to its cellular site of action. The first generation of heterobifunctional cross-linkers used to bind the toxin to the monoclonal antibody generated disulfide bonds that were unstable in vivo. This problem was solved in part by the synthesis of more stable cross-linkers, which used phenyl or methyl groups, or both, adjacent to the disulfide bond to restrict access to the bond.

The activity of an immunotoxin is initially assessed by measuring its ability to kill cells with target antigens on their surfaces. Because toxins act within the cells, receptors and other surface proteins that naturally enter cells by endocytosis usually make good targets for immunotoxins, but surface proteins that are fixed on the cell surface do not. However, if several antibodies recognizing different epitopes on the same cell surface protein are available, it is useful to test them all. This is because some, perhaps by producing a conformational change in the target proteins' structure, may induce its internalization or direct its intracellular routing to an appropriate location for toxin translocation. May et al., Cell Immunol., 135, 490 (1991). Also, it is possible to induce internalization of a target surface protein if the immunotoxin contains a form of a toxin in which the binding of the toxin moiety to its receptor, although weakened by chemical modification, still occurs and promotes internalization since toxin receptors are efficiently internalized. Willingham et al., Proc. Natl. Acad. Sci. USA, 84, 2474 (1987).

Several immunotoxins have been developed and approved for human trials. Two different kinds of trials have been conducted. The first involves the ex vivo addition of immunotoxins to harvested bone marrow to eliminate containing tumor cells before reinfusion in patients undergoing autologous bone marrow transplantation. A variety of antibodies, linked to ricin or ricin A chain, including anti-CD5 and anti- CD7, have been used for this purpose. Uckun et al., Blood, 76, 1723 (1990). The second kind of trial involves the parenteral administration of immunotoxins, either regionally (such as to the peritoneal cavity) or systemically, to patients with cancer. These have been primarily Phase 1 and 2 trials in patients in which conventional treatments have failed, and the patients have a large tumor burden. So far, the antibodies used for the preparation of immunotoxins to treat carcinomas or other solid tumors have been found to react with important normal human tissues (such as neural tissue and bone marrow) and produce dose-limiting toxicity without significant clinical responses. See, for example, Gould et al., J. Natl. Cancer Inst., 81 775 (1989).

b. Fusion Proteins

Fusion proteins are hybrid modified PAP polypeptides made by recombinant DNA technology that are designed to selectively target one or more modified PAP polypeptides of the present invention to a cell infected by a virus. Fusion proteins are synthesized by the fusion of a targeting moiety that binds to a receptor on a cell to a modified PAP of the invention. The cytotoxic agent is preferably a portion of a bacterial or plant toxin. The activity of these fusion proteins depends not only on the toxin utilized, but also on efficient binding of antibody to antigen, endocytosis, and intracellular release of functional ribosome inactivating proteins.

c. Targeting Moieties

Since it is established that many cells infected with a virus overproduce cytokine receptorsreceptors, the targets for this type of therapy can be growth factor receptors, differentiation antigens, or other less characterized cell surface antigens. Thus, effective targeting moieties include, but are not limited to, cytokines, cytokine subunits, antibodies or antibody subunits. Specifically, as used herein the term “targeting moiety” is defined to mean all monoclonal antibodies, monoclonal antibody fragments, single chain variable region polypeptides, and cytokines used in the production of immunotoxins and fusion toxins. Useful examples of targeting moieties include, but are not limited to, a monoclonal antibody, monoclonal antibody fragment, or single chain variable region polypeptide directed against the CD2, CD3, CD4, CD5, CD7, CD 13, CD 14, CD 19, CD22, CD24, CD33, CD40, CD45, CD72, TXU.1, NXU.1, TP-1, or TP-3 antigen. Furthermore, the targeting moiety of the present invention may be a cytokine. If the targeting moiety is a cytokine, preferred cytokines include, but are not limited to, GM-CSF, IL-2, IL 3, IL-4, IL-6, IL-7, IL-8, IL-9, IL-10, IL-12, EGF, FGF, PDGF, or NGF. The targeting moiety can be a HIV specific antigen, such as gp120, or other HIV specific antigens that can be presented on the surface of a host cell. The targeting moiety can also be CCR5.

One immunoconjugate of PAP, TXU (anti-CD7)-PAP has been described as having favorable properties for treatment of HIV in humans (Uckun et al., J. Pharmacol. Exp. Ther., 1999, 291(3):1301-7). Thus, TXU-modified PAP is expected to be effective.

1. Antibodies

Monoclonal antibodies (MoAbs) are produced by the fusion of spleen lymphocytes with malignant cells (myelomas) of bone marrow primary tumors. Milstein, Sci. Am., 243, 66 (1980). The procedure yields a hybrid cell line, arising from a single fused cell hybrid, or clone, which possesses characteristics of both the lymphocytes and myeloma cell lines. Like the lymphocytes (taken from animals primed with sheep red blood cells as antigens), the fused hybrids or hybridomas secrete antibodies (immunoglobulins) reactive with the antigen. Moreover, like the myeloma cell lines, the hybrid cell lines are immortal. Specifically, whereas antisera derived from vaccinated animals are variable mixtures of antibodies which cannot be identically reproduced, the single-type of immunoglobulin secreted by a hybridoma is specific to one and only one determinant on the antigen, a complex molecule having a multiplicity of antigenic molecular substructures, or determinants (epitopes). Hence, monoclonal antibodies raised against a single antigen may be distinct from each other depending on the determinant that induced their formation. However, all of the antibodies produced by a given clone are identical. Furthermore, hybridoma cell lines can be reproduced indefinitely, are easily propagated in vitro and in vivo, and yield monoclonal antibodies in extremely high concentration.

Monoclonal antibodies have largely been applied clinically to the diagnosis and therapy of cancer and the modulation of the immune response to produce immunosuppression for treatment of autoimmune and graft versus host diseases (GVHD) and for prevention of allograft rejection. Human monoclonal antibodies have also been applied clinically against cytomegalovirus, Varicella zoster virus, and the various specific serotypes of Pseudomonas aeruginosa, Escherichia coli, and Klebsiella pneumoniae.

Antibodies and their fragments can also be genetically engineered to have more rapid clearance. This is desirable when a monoclonal antibody is conjugated to a radionucleotide for use in radioimmunoscanning. For example, antigen-binding fragment (Fab), F(ab′).sub.2, or single chain Fv fragments of monoclonal antibodies have survival half lives of less than 5 hours. Rapid turnover can also be accomplished by the deletion of the CH.sub.2 domain as demonstrated for an antibody reactive with the disaloganglioside GD2 expressed on human tumors of neuroectodermal origin. Mueller et al., Proc. Natl. Acad. Sci. USA, 87, 5702 (1990).

Furthermore, due to their large size, intact antibodies and the corresponding antibody-toxin conjugates are restricted in their ability to migrate from the vascular regions, are heterogenous as immunoconjugates (which can result in linkage of several toxin molecules to one immunoglobulin molecule), and their production is expensive and very labor intensive. See, for example, U.S. Pat. No. 4,831,117 to Uckun and U.S. Pat. No. 4,671,958 to Rodwell et al., the teachings of which are herein incorporated by reference. Again, genetic engineering has been used for the expression of the light and heavy chain variable regions of antibodies in bacteria as single chain Fv (scFv) fragments in an attempt to improve on the efficacy of antibodies and their corresponding immunoconjugates. Pastan et al., Science, 254, 1173 (1991). In general, these molecules have been insoluble and need to be denatured and refolded before binding activity can be detected. One problem with production of antibody binding domains in this manner is that high affinity antibody binding cannot be successfully reconstituted in all instances. The parameters that govern the ability of an antibody to yield scFv that can bind to its target are unknown, thus necessitating the direct cloning and analysis of the candidate antibody gene segments.

4. Production and Purification of Biotherapeutic Agents

a. Immunoconjugates

Preferred antibody-PAP immunotoxins for use in the method are formed by linking an effective cytotoxic amount of PAP molecules to each molecule of antibody. For example, a reagent useful in the practice of the invention is an about 1:1 mixture of antibody-PAP having one to three PAP molecules per antibody molecule, respectively. However, antibody molecules can be linked to effective amounts of PAP by other means disclosed in the art, including those taught in U.S. Pat. Nos. 4,363,758, Masuho et al.; 5,167,956, Neville, Jr. et al. and 4,340,535, Voisin et al. For example, in addition to N-succinimidyl 3-2-pyridyldithio)propionate (SPDP), 4-succinimidyloxycarbonyl-methyl-(2-pyridyldithio)-toluene (SMPT) and N-succimidyl 6-[3-(2-pyridyldithio)propionamido]hexanoate (LC-SPDP) may be used as linking agents.

b. Fusion Proteins

DNA encoding PAP can be derived or isolated from a suitable source, such as a plant source, subsequently chemically altered in vitro, and later introduced into target host cells, such as cells derived from animal, plant, insect, yeast, fungal or bacterial sources. An example of PAP DNA “derived” from a source, would be a DNA sequence that is identified as a useful fragment encoding PAP, or a fragment, mutant or variant thereof, and which is then chemically synthesized in essentially pure form. An example of such PAP DNA “isolated” from a source would be a useful DNA sequence that is excised or removed from plant cells by chemical means, e.g, by the use of restriction endonucleases, so that it can be further manipulated, e.g., amplified, for use in the invention, by the methodology of genetic engineering.

Therefore, DNA encoding recombinant PAP includes completely synthetic DNA sequences, semi-synthetic DNA sequences, native DNA sequences isolated from plant cells, and DNA sequences derived from introduced RNA, as well as mixtures thereof. Generally, the recombinant DNA sequence is not originally resident in the genome of the host target cell which is the recipient of the DNA, nor is it resident in the genome and not expressed.

The vector comprising recombinant PAP DNA sequence, used for transformation herein, may be circular or linear, double-stranded or single-stranded. Generally, the DNA sequence is in the form of chimeric DNA, such as plasmid DNA, that can also contain coding regions flanked by control sequences which promote the expression of the recombinant DNA present in the resultant transformed host cells. For example, the recombinant DNA may itself comprise a foreign promoter that is active in cells, or may utilize a promoter already present in the genome that is the transformation target. Such promoters include the GAL1 promoter, the T7 promoter, the Lac UV5 promoter, the CMV promoter, as well as the SV 40 late promoter and retroviral LTRs (long terminal repeat elements). Aside from recombinant DNA sequences that serve as transcription units for PAP or portions thereof, a portion of the recombinant DNA may be untranscribed, serving a regulatory or a structural function.

“Control sequences” is defined herein to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a PAP polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The recombinant transformation vector will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as ura, neo, hpt, dhfr, bar, aroA, dapA and the like.

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis. Expression of the reporter gene is assayed at a suitable time after the DNA has been introduced into the recipient cells.

Other elements functional in the host cells, such as introns, enhancers, polyadenylation sequences and the like, may also be a part of the recombinant PAP. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The general methods for constructing recombinant DNA which can transform target cells are well known to those skilled in the art, and the same compositions and methods of construction may be utilized to produce the recombinant PAP useful herein. For example, J. Sambrook et al., Molecular Cloning: A Laboratory Manual, Cold Spring Harbor Laboratory Press (2d ed., 1989), provides suitable methods of construction.

The recombinant PAP can be readily introduced into the target cells by transfection with an expression vector comprising cDNA encoding PAP, for example, by the modified calcium phosphate precipitation procedure of C. Chen et al., Mol. Cell. Biol., 7, 2745 (1987). Transfection can also be accomplished by lipofectin, using commercially available kits, e.g., provided by BRL.

Suitable host cells for the expression of the recombinant PAP are derived from multicellular organisms, such as yeasts, insects and plants. Such host cells are capable of complex processing and glycosylation activities. In principle, any higher eukaryotic cell culture is functional, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. See, e.g., Luckow et al., Bio/Technolog, 6, 47 (1988); Miller et al., in Genetic Engineering, J. K. Setlow et al., eds., Vol. 8 (Plenum Publishing, 1986), pp. 277-279; and Maeda et al., Nature, 315, 592 (1985). A variety of viral strains for transfection are publicly available, e.g., the L-1 variant of Autographa californica NPV and the Bm-5 strain of Bombyx mori NPV, and such viruses may be used, preferably for transfection of Spodoptera frugiperda cells.

The baculovirus-insect cell system is often used because it closely mimics mammalian expression of proteins, in that proteins can be produced with appropriate post-translational modifications (A. Angermann et al, Eur. J. Biochem., 206, 225 (1992); M. D. Summers et al., A Manual of Methods for baculovirus Vectors and Insect Cell Culture Procedures of MicroGene System Inc., New Haven, Conn. (1988); D. R. O'Reilly et al., Baculovirus Expression Vectors: Laboratory Manual, Oxford Univ. Press, N.Y. (1994)).

Recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. For example, see Lawn et al., Nucleic Acids Res., 9, 6103 (1981), and Goeddel et al., Nucleic Acids Res., 8, 4057 (1980).

“Southern analysis” or “Southern blotting” is a method by which the presence of DNA sequences in a restriction endonuclease digest of DNA or DNA-containing composition is confirmed by hybridization to a known, labeled oligonucleotide or DNA fragment. Southern analysis typically involves electrophoretic separation of DNA digests on agarose gels, denaturation of the DNA after electrophoretic separation, and transfer of the DNA to nitrocellulose, nylon, or another suitable membrane support for analysis with a radiolabeled, biotinylated, or enzyme-labeled probe as described in sections 9.37-9.52 of Sambrook et al., supra.

“Northern analysis” or “Northern blotting” is a method used to identify RNA sequences that hybridize to a known probe such as an oligonucleotide, DNA fragment, cDNA or fragment thereof, or RNA fragment. The probe is labeled with a radioisotope such as 32-P, by biotinylation or with an enzyme. The RNA to be analyzed can be usually electrophoretically separated on an agarose or polyacrylamide gel, transferred to nitrocellulose, nylon, or other suitable membrane, and hybridized with the probe, using standard techniques well known in the art such as those described in sections 7.39-7.52 of Sambrook et al., supra.

“Polymerase chain reaction” or “PCR” refers to a procedure or technique in which amounts of a preselected piece of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences, specific DNA sequences from total genomic DNA, and cDNA transcribed from total cellular RNA, bacteriophage or plasmid sequences, and the like. See generally Mullis et al., Cold Spring Harbor Symp. Ouant. Biol., 51, 263 (1987); Erlich, ed., PCR Technology, (Stockton Press, N.Y., 1989).

“Stringent conditions” are those that (1) employ low ionic strength and high temperature for washing, for example, 0.015M NaCl/0.0015M sodium citrate (SSC); 0.1% sodium lauryl sulfate (SDS) at 50.degree. C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42.degree. C. Another example is use of 50% formamide, 5.times. SSC (0.75M NaCl, 0.075M sodium citrate), 50 mM sodium phosphate (pH 6.8), 0.1% sodium pyrophosphate, 5.times. Denhardt's solution, sonicated salmon sperm DNA (50.mu.g/ml), 0.1% SDS, and 10% dextran sulfate at 42.degree. C., with washes at 42.degree. C. in 0.2 times. SSC and 0.1% SDS.

When recombinant PAP is expressed in a recombinant cell other than one of human origin, the PAP polypeptide is completely free of proteins or polypeptides of human origin. However, it is necessary to purify PAP polypeptide from recombinant cell proteins or polypeptides to obtain preparations that are substantially homogeneous as to native PAP. For example, the culture medium or lysate can be centrifuged to remove particulate cell debris. The membrane and soluble protein fractions are then separated. The PAP polypeptide may then be purified from the soluble protein fraction and, if necessary, from the membrane fraction of the culture lysate. PAP polypeptide can then be purified from contaminant soluble proteins and polypeptides by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; or ligand affinity chromatography.

Once isolated from the resulting transgenic host cells, derivatives and variants of the PAP polypeptide can be readily prepared. One or more of the residues of the PAP polypeptide can be altered, so long as the antiviral activity is retained. Conservative amino acid substitutions are preferred—that is, for example, aspartic-glutamic as acidic amino acids; lysinelarginine/histidine as basic amino acids; leucine/isoleucine, methionine/valine as hydrophobic amino acids; serine/glycine/alanine/threonine as hydrophilic amino acids. Additionally, salts of carboxyl groups of the polypeptide may be prepared in the usual manner by contacting the polypeptide with one or more equivalents of a desired base, such as, for example, a metallic hydroxide base, e.g., sodium hydroxide; a metal carbonate or bicarbonate base, such as, for example, sodium carbonate or sodium bicarbonate; or an amine base such as, for example, triethylamine, triethanolamine, and the like.

Furthermore acid addition salts of the polypeptides may be prepared by contacting the polypeptide with one or more equivalents of the desired inorganic or organic acid, such as for example, hydrochloric acid.

Esters of carboxyl groups of the polypeptides may be prepared by any of the usual means known in the art for converting a carboxylic acid or precursor to an ester. One preferred method for preparing esters of the present polypeptides, is to cleave the completed polypeptide from the resin in the presence of the desired alcohol either under basic or acidic conditions, depending upon the resin. Thus, the C-terminal end of the polypeptide, when freed from the resin, is directly esterified without isolation of the free acid.

Amides of the isolated polypeptides may also be prepared by techniques well known in the art for converting a carboxylic acid group or precursor, to an amide. A preferred method for amide formation at the C-terminal carboxyl group is to cleave the polypeptide from a solid support with an appropriate amine, or to cleave in the presence of an alcohol, yielding an ester, followed by aminolysis with the desired amine.

N-acyl derivatives of an amino group of the present polypeptides may be prepared by utilizing an N-acyl protected or unprotected peptide. 0-acyl derivatives may be prepared, for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

The following references describe preparation of polypeptide analogs which include non-peptidyl bonds to link amino acid residues. Spatola, Vega Data, 1, 3 (1983); Hudson et al., Int J. Pept. Prot. Res., 14, 177 (1979); Spatola in “Chemistry and Biochemistry of Amino Acids, Peptides and Proteins”, B. Weinstein, eds., Marcel Dekker, N.Y., p. 267 (1983); Spatola et al., Life Sci., 38 1243 (1986); Almquist et al., J. Med. Chem., 23, 1392 (1980); Holladay et al., Tetrahedron Letters, 24, 4401 (1983).

To prepare the fusion protein of the present invention, the PAP gene can be linked either to the gene that encodes the mature form of a cytokine suitable for use in the present invention or a novel genetically engineered monoclonal antibody subunit, e.g., the Fv fragment, preferably at the site of the flexible molecular hinge. In addition, a synthetic DNA sequence encoding a short Ser-(Gly)4-Ser-Met intervening linker can be inserted at the hinge site separating the PAP and cytokine or MoAb scFv moieties to insure that the binding domains would be available for participation in high affinity receptor binding. This rational drug design of recombinant polypeptide cytotoxins is intended to preserve essential structure-function relationships identified in crystallographic analyses of both the PAP and cytokine or antibody molecules. Rambaldi et al., Blood, 81, 1376 (1993).

The pET11d expression vector (Novagen, Inc.; 597 Science Drive, Madison, Wis. 53711) employed for the production of recombinant polypeptide cytotoxins in E. coli contains a hybrid bacteriophage T7 promoter with a 3′lac operator sequence fusion and an internal copy of lad to suppress basal expression, an efficient Shine-Dalgarno sequence for translational efficiency, and an NcoI cloning site for the insertion of recombinant scFv, dsFv, and toxin gene fusions. The gene encoding the bacteriophage T7 polymerase gene is incorporated by lysogeny into the genome of an E. coli expression host and is under the control of the lac UV5 promoter. For example, the pLysS gene in the HMS 174(de3)plysS host produces a low amount of the T7 lysozyme, a natural inhibitor of T7 RNA polymerase, to provide additional stringency of gene expression regulation. Expression of the biotherapeutic agents from within pET11d expression vectors can be induced by the addition of isopropylthiogalactoside (IPTG) to the media containing the E. coli expression host.

The biotherapeutic agents are individually expressed in a host such as HMS174(de3)plysS and the soluble product is recovered from cells disrupted by freeze-thaw cycles and sonication. The soluble fraction containing the recombinant polypeptide cytotoxin is subsequently purified through sequential filtration, anti-toxin immunoaffinity chromatography, filtration and dialysis, anion exchange high performance liquid chromatography, additional filtration endotoxin removal resins, and final filtration and dialysis. Insoluble product can be rendered to a soluble form for purification by dissolution in 7M guanidine HCl with a slow renaturation under controlled conditions to a physiological buffer such as phosphate buffered saline.

F. Modes of Administration of the Biotherapeutic Agents

The present modified PAP polypeptides can be formulated as pharmaceutical compositions and administered to a human or other mammal afflicted with a condition treatable by these agents, alone or in combination in a unit dosage form comprising an effective amount of one or more of these agents in combination with a pharmaceutically acceptable carrier or vehicle.

1. Dosage Forms

It is preferred that modified PAP polypeptides of the present invention be parenterally administered, i.e., intravenously, or subcutaneously by infusion or injection. Solutions or suspensions of the modified PAP polypeptides can be prepared in water, or isotonic saline, such as PBS, optionally mixed with a nontoxic surfactant. Dispersions can also be prepared in glycerol, liquid polyethylene glycols, DMA, vegetable oils, triacetin, and mixtures thereof. Under ordinary conditions of storage and use, these preparations may contain a preservative to prevent the growth of microorganisms. Additionally, more specific delivery of the modified PAP polypeptides to the lungs may be accomplished via aerosol delivery systems. The pharmaceutical dosage form suitable for aerosol delivery can include adipot formulations such as a liposome of suitable size.

The pharmaceutical dosage form suitable for injection or infusion use can include sterile aqueous solutions or dispersions or sterile powders comprising the modified PAP polypeptides which are adapted for the extemporaneous preparation of sterile injectable or infusible solutions or dispersions. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, and liquid polyethylene glycols, and the like), vegetable oils, nontoxic glycerol esters, lipids (for example, dimyristoyl phosphatidyl choline) and suitable mixtures thereof The proper fluidity can be maintained, for example, by the formation of liposomes, by the maintenance of the required particle size in the case of dispersion or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers or sodium chloride. Prolonged absorption of the injectable compositions can be brought about by the inclusion in the compositions of agents delaying absorption, for example, aluminum monostearate hydrogels and gelatin.

Sterile injectable or infusable solutions are prepared by incorporating the modified PAP polypeptides in the required amount in the appropriate solvent with various of the other ingredients enumerated above, and as required, followed by filter sterilization. In the case of sterile powders for the preparation of sterile injectable or infusable solutions, the preferred methods of preparation are vacuum drying and the freeze drying techniques, which yield a powder of the active ingredient plus any additional desired ingredient present in the previously sterile-filtered solutions.

Furthermore, suitable formulations for the modified PAP polypeptides of the present invention include those suitable for oral, rectal, nasal, topical (including, ocular, and sublingual) or vaginal administration or in a form suitable for administration by inhalation or insufflation. The formulations may be prepared by any of the methods well known in the art of pharmacy. Such methods include the step of bringing into association the modified PAP polypeptides with liquid carriers or finely divided solid carriers or both and then, if necessary, shaping the product into the desired formulation.

Pharmaceutical formulations suitable for oral administration may conveniently be presented as discrete units such as capsules, sachets, or tablets, each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The modified PAP polypeptides may also be formulated for intra-nasal or ocular administration. In this form of administration, the active ingredient may be used as a liquid spray or dispersible powder or in the form of drops. Drops, for example, eyedrops, may be formulated with an aqueous or non-aqueous base also comprising one or more dispersing agents, solubilizing agents or suspending agents. Liquid sprays are conveniently delivered from pressurized packs.

For administration by inhalation, the biot modified PAP polypeptides are conveniently delivered from an insufflator, nebulizer or a pressurized pack or other convenient means of delivering an aerosol spray. Pressurized packs may comprise a suitable propellant such as dichlorodifluoromethane, trichlorofluromethane, dichlorotetrafluoroethane, carbon dioxide or other suitable gas. In the case of a pressurized aerosol, the dosage unit may be determined by providing a valve to deliver a metered amount.

Alternatively, for administration by inhalation of insufflation, the modified PAP polypeptides may take the form of a dry powder composition, for example, a powder mix of the compound or a suitable powder base such as lactose or starch. The powder composition may be presented in unit dosage form in, for example, capsules or cartridge or e.g., gelatin or blister packs from which the powder may be administered with the aid of an inhaler of insufflator.

Additionally, the modified PAP polypeptides are well suited to formulation or controlled release dosage forms. The formulations can be so constituted that they release the active dry ingredient only or preferably in a particular physiological location, optionally over a period of time. The coatings, envelopes, and protective matrices may be made, for example, from polymeric substances or waxes. The compounds can also be delivered via patches for transdernal delivery, subcutaneous implants, infusion pumps or via release from implanted depot sustained release dosage forms.

2. Dosages

The dosage of the modified PAP polypeptides in said composition can be varied widely, in accord with the size, age and condition of the patient and the target cancer. Based on animal data, it is expected that the dosage can be varied between 0.025 mg/kg/day and I mg/kg/day, administered over a period of about 3 to 5 days.

The invention will be further described by reference to the following detailed examples.

### EXAMPLES

### Example 1

### Rational Design of Mutant PAP Proteins: Molecular Modeling

Molecular modeling studies for the rational design of recombinant PAP proteins were performed, as described in detail previously (Rajamohan F, et al., 2000, Journal of Biological Chemistry, 275(5):3382-3390; Rajamohan, F., et al., 2001, Journal of Biological Chemistry, 276(26): 24075-81; Rajamohan, F., et al., 2001, Biochemistry, 40:9104-14). The molecular model of the PAP-ribosome (large subunit) complex was derived from the 2.4-Å crystal structure of the large ribosomal subunit from Haloarcula marismortui (Protein Data Bank access code 1FFK) (Ban et al., (2000) Science 289:905-20.) and the crystal structure of PAP-nucleotide complex (access code 1pag).

Structure-Based Design of Nontoxic Mutant PAP Proteins with Potent Anti-HIV Activity.

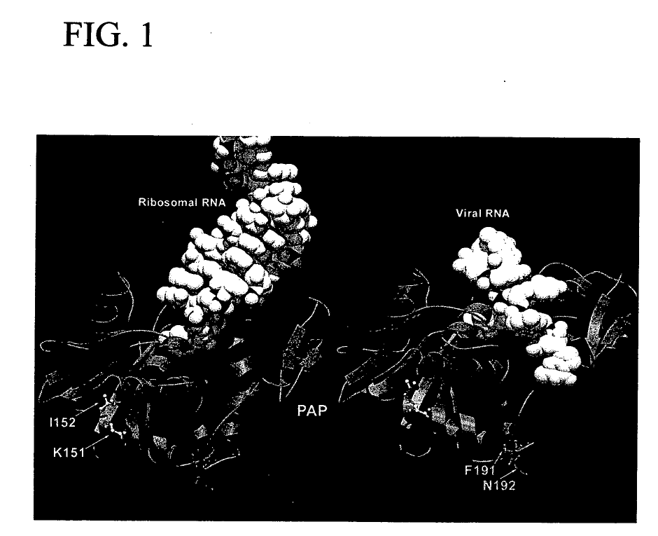

Molecular modeling studies indicated that ribosomal RNA and HIV-1 RNA adopt distinctly different binding modes in their interactions with PAP. In a systematic search for specific mutations that might result in selective enhancement of the anti-HIV activity of PAP, we noticed that the residue I152 on the opposite side of the active site is buried (FIG. 1). Residues K151 and I152 are located on helix α4 which is followed by a loop and helix α5. The side chain of I152 is mostly buried in hydrophobic contact (approximately 3-4Å) with those of F158 (from the loop), T162 and F166 (helix α5), and Y76 from the middle β strand. In contrast, the side chain of K151 is mostly exposed and may form a hydrogen bond with N148 from the same helix. Residue 152 is approximately 20 Å from the catalytic residue R179 and situated on the opposite side of the PAP active site.

It is known that alanine substitution of hydrophobic residues on an alpha helix would generally not disrupt a helical conformation (Bell J A, et al., 1992, Biochemistry, 31(14):3590-6). In many cases, a large-to-small mutation such as I152A may, in fact, be structurally favored for a helical conformation (Zhang X J, et al., 1992, Protein Sci, 1(6):761-76). However, in light of multiple van der Waals contacts between I152 and surrounding residues, I152A mutation could potentially create a cavity in an otherwise tightly packed hydrophobic region. The bulky isoleucine side chain within the core of the protein confers greater hydrophobic stabilization than is the case for the smaller alanine side chain. The Ile to Ala substitution that can create a large cavity would be especially destabilizing because it would result in a loss of both hydrophobic and van der Waals interactions. Consequently, the surrounding residues would relax and reduce the volume of the putative cavity. Such mutations may cause local conformational instability and lead to a significant conformational change (Matthews B W., 1995, Adv Protein Chem, 46:249-78). The side chain of the residue could rotate into a radically different orientation and permit repacking of the core as exemplified in the S117 mutation observed in T4 lysozyme. Such repacking is associated with adjustments of both the main chain and side chains. The backbone of a protein that contains mutated residues could deviate from the natural conformation. Such conformational changes could reach and affect the dynamic behavior of the catalytic residues that are farther than 25 Å away, as previously shown for the G93A mutation in superoxide dismutase (SOD) (Yim H S, et al., 1997, J Biol. Chem, 272(14):8861-3; Liu R, et al., 1999, Radiation Research, 151, 133-141). We and other groups have long recognized the dramatic effect that this kind of mutation may affect the enzyme activity (not substrate binding) and have been actively pursuing the concept in our protein engineering.

We have previously modeled the complex structure of PAP with ribosome, based on the crystal structures of PAP and ribosome, and the complex structure of PAP with an HIV viral RNA substrate (Kurinov IV, et al., 1999, Protein Science, 8(9): 1765-1772). The proposed structural model is in good agreement with our mutation studies of residues involved in the active site and the extended substrate-binding site (Rajamohan F, et al., 2000, Journal of Biological Chemistry, 275(5):3382-3390). Based on our modeling analyses, we have recognized that the binding mode of ribosome is distinctly different from that of viral RNA as shown in FIG. 1. Our modeling studies indicated that a substitution of this isoleucine with a smaller residue such as alanine (I152A) would likely create a cavity causing a repacking of the core and considerable conformational changes of the active site that would be selectively unfavorable for PAP-mediated depurination of ribosomal RNA, but possibly would not affect the catalytic depurination of HIV-1 RNA.

A double mutation of both I152 and K151 residues was predicted to result in a more substantial loss of ribosome inhibitory activity than single mutations involving only one of these two residues. Residues F191 and N192 are located in a more flexible environment on the C-terminal end of helix 6 approximately 20 Å away from the catalytic residue R179 (FIG. 1).

Similar to the binding environment of I152, the side chain of F191 is buried and is in hydrophobic contact with the side chains of I142, I13, Y16, and the hydrophobic portion of K188, all of which are situated on nearby helices. The side chain of N192 is mostly exposed and shows no significant contact with nearby residues. We postulated that substitutions of these residues with alanine or glycine would cause conformational changes in the active site leading to a moderately reduced activity towards ribosomal RNA (but not HIV-1 RNA) substrates, albeit to a much lesser degree than the I152A mutation. However, none of the K151, I152, F191, or N192 mutations would cause an impairment in the ability of the respective PAP proteins to bind ribosomes, or the ribosomal protein L3 which interacts with the partially exposed half of the active site cleft (FIG. 1).

FIG. 1.

Ribbon and Space-filling Representation of PAP (Gray Ribbons) Models Complexed with Ribosomal RNA (Left, in White) and Viral RNA (Right, in White) Molecules.

Residues I152 and K151 as shown in stick model are mutated in FLP-102. Residues F191 and N192 as shown in stick model are mutated in FLP-105. These residues are located on the opposite side of the PAP active site and the mutation of these residues is not predicted to affect the binding of ribosomal RNA or HIV-1 viral RNA substrates. However, residue I152 is buried and the large-to-small mutation of Ile to Ala would create a cavity and permit repacking of the core, thus leading to considerable conformational changes of catalytic residues and differentially affecting the activity of PAP against the ribosome substrate and the viral RNA.

### Example 2

### Engineering, Expression and Purification of Recombinant Mutant PAP Protein

Engineering

Recombinant wild-type PAP (PBS-PAP) was obtained by subcloning the PAP-I gene (amino acids 22 to 313) into the pBluescript SK⁻ expression vector (Rajamohan F, et al., Protein Expression and Purification, 16(2): 359-368). The PAP-I gene was amplified by polymerase chain reaction (PCR) using the following primers PAP-Bam (5′ CGC GGA TCC AGT GAA TAC AAT CAT CTA CAT GTT GGA AGT ACC 3′) (SEQ ID NO: 2), which introduces a BamHII site at the N-terminus, and PAP-H3 (5′ GCC TCT TAT TTA AGC TTT ATA ATA TAG TTG GAG 3′) (SEQ ID NO: 3), which introduces a HindII site at the C-terminus. A uracil-containing template of PAP was obtained by transforming E. coli CJ236 with the recombinant plasmid PBS-PAP. The oligonucleotides used for site-directed mutagenesis were synthesized on the 200 nmol scale and HPLC purified by Biosynthesis Inc. (Lewisville, Tex.). A site-directed mutagenesis procedure was performed as described in the manufacturer's manual using the Mutagene M13 In vitro Mutagenesis Kit (Bio-Rad, Hercules, Calif.). DNA sequencing was carried out by the method of Sanger et al. (Proc Natl Acad Sci USA 74(12), 5463-7, 1977), following the manufacturer's instructions (U.S. Biochemical Corp. Cleveland, Ohio). Chemicals and restriction enzymes were purchased from Roche Molecular Biochemicals (Indianapolis, Ind.).

Expression and Purification

Wild-type and mutant PAP proteins were expressed in E. coli MV1190 as inclusion bodies, and then isolated, solubilized, and refolded, as described previously (Rajamohan F, et al., Protein Expression and Purification, 16(2): 359-368). The refolded proteins were analyzed by sodium dodecyl sulfate-12% polyacrylamide gel electrophoresis (SDS-12% PAGE). Protein concentrations were quantitated from the gel using bovine serum albumin as a standard.

Immunoblot Analysis

Protein samples were resolved on a SDS-12% PAGE and transferred onto a polyvinylidene difluoride membrane (Bio-Rad) using Bio-Rad Trans-Blot apparatus, as described previously (Rajamohan F, et al., Protein Expression and Purification, 16(2): 359-368). The membrane was immunoblotted using a rabbit anti-PAP serum (1:2000 dilution) and a horseradish peroxidase-conjugated goat anti-rabbit IgG (Sigma Chemical Co., St. Louis, Mo.) as the primary and secondary antibodies, respectively. The blot was developed using 3,3′-diaminobenzidine (Sigma) as the colorimetric indicator for peroxidase activity.

Results

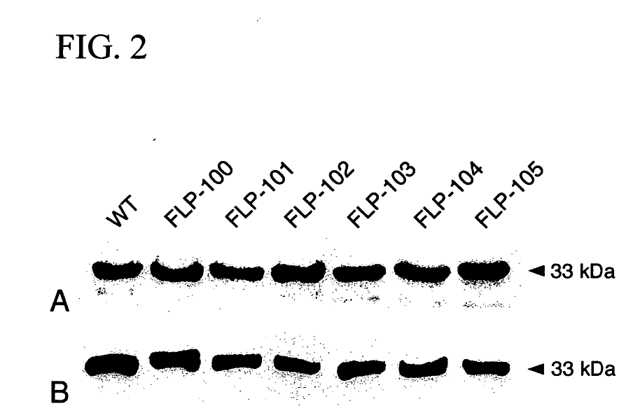

As described above, the recombinant PAP mutants with alanine substitutions of I152, K151, and F191, and glycine substitution of N192 were constructed using site-directed mutagenesis (Rajamohan F, et al., Protein Expression and Purification, 16(2): 359-368; Rajamohan F, et al., 2000, Journal of Biological Chemistry, 275(5):3382-3390). The mutant proteins FLP-100 (K151A), FLP-101 (I152A), FLP-102 (K151A, I152A), FLP-103 (F191A), FLP-104 (N192G), and FLP-105 (F191A, N192G) were expressed in the E. coli strain, MV1190, as inclusion bodies, purified, solubilized, refolded, and analyzed by SDS-PAGE (FIG. 2A, 5 μg per lane). Each of the mutant PAP proteins had an apparent molecular mass of 33 kDa similar to that of the recombinant wild-type PAP (FIG. 2A). The refolded recombinant wild-type and mutant proteins were highly immunoreactive with anti-PAP serum by Western blot analysis (FIG. 2B, 5 μg per lane).

TABLE 2

(SEQ ID NO: 1)

```
CTATGAAGTC GGGTCAAAGC ATATACAGGC TATGCATTGT TAGAAACATT GATGCCTCTG
ATCCCGATAA ACAATACAAA TTAGACAATA AGATGACATA CAAGTACCTA AACTGTGTAT
GGGGGAGTGA AACCTCAGCT GCTAAAAAAC GTTGTAAGAA AAAAAGAAAG TTGTGAGTTA
ACTACAGGGC GAAAGTATTG GAACT
              (1)
AGCTAGTAGGAAGGGAAG ATG AAG TCG ATG CTT GTG GTG ACA ATA TCA ATA TGG CTC
              Met Lys Ser Met Leu Val Val Thr Ile Ser Ile Trp Leu
                                (67)
ATT CTT GCA CCA ACT TCA ACT TGG GCT GTG AAT ACA ATC ATC TAC AAT GTT GGA AGT
Ile Leu Ala Pro Thr Ser Thr Trp Ala Val Asn Thr Ile Ile Tyr Asn Val Gly Ser
                                            (1)
  (100)
ACC ACC ATT AGC AAA TAC GCC ACT TTT CTG AAT GAT CTT CGT AAT GAA GCG AAA
Thr Thr Ile Ser Lys Tyr Ala Thr Phe Leu Asn Asp Leu Arg Asn Glu Ala Lys
(10) (20)
GAT CCA AGT TTA AAA TGC TAT GGA ATA CCA ATG CTG CCC AAT ACA AAT ACA AAT
Asp Pro Ser Leu Lys Cys Tyr Gly Ile Pro Met Leu Pro Asn Thr Asn Thr Asn
  (30) (40)
CCA AAG TAC GTG TTG GTT GAG CTC CAA GGT TCA AAT AAA AAA ACC ATC ACA CTA
Pro Lys Tyr Val Leu Val Glu Leu Gln Gly Ser Asn Lys Lys Thr Ile Thr Leu
            (50) (60)
ATG CTG AGA CGA AAC AAT TTG TAT GTG ATG GGT TAT TCT GAT CCC TTT GAA ACC
Met Leu Arg Arg Asn Asn Leu Tyr Val Met Gly Tyr Ser Asp Pro Phe Glu Thr
                    (70) (80)
AAT AAA TGT CGT TAC CAT ATC TTT AAT GAT ATC TCA GGT ACT GAA CGC CAA GAT
Asn Lys Cys Arg Tyr His Ile Phe Asn Asp Ile Ser Gly Thr Glu Arg Gln Asp
                            (90) (100)
GTA GAG ACT ACT CTT TGC CCA AAT GCC AAT TCT CGT GTT AGT AAA AAC ATA AAC TTT
Val Glu Thr Thr Leu Cys Pro Asn Ala Asn Ser Arg Val Ser Lys Asn Ile Asn Phe
                                        (110)
GAT AGT CGA TAT CCA ACA TTG GAA TCA AAA GCG GGA GTA AAA TCA AGA AGT CAG
Asp Ser Arg Tyr Pro Thr Leu Glu Ser Lys Ala Gly Val Lys Ser Arg Ser Gln
(120) (130)
GTC CAA CTG GGA ATT CAA ATA CTC GAC AGT AAT ATT GGA AAG ATT TCT GGA GTG
Val Gln Leu Gly Ile Gln Ile Leu Asp Ser Asn Ile Gly Lys Ile Ser Gly Val
        (140) (150)
ATG TCA TTC ACT GAG AAA ACC GAA GCC GAA TTC CTA TTG GTA GCC ATA CAA ATG
Met Ser Phe Thr Glu Lys Thr Glu Ala Glu Phe Leu Leu Val Ala Ile Gln Met
                  (160) (170)
GTA TCA GAG GCA GCA AGA TTC AAG TAC ATA GAG AAT CAG GTG AAA ACT AAT TTT
Val Ser Glu Ala Ala Arg Phe Lys Tyr Ile Gku Asn Gln Val Lys Thr Asn Phe
                          (180) (190)
AAC AGA GCA TTC AAC CCT AAT CCC AAA GTA CTT AAT TTG CAA GAG ACA TGG GGT
Asa Arg Ala Phe Asn Pro Asn Pro Lys Val Leu Asn Leu Gln Glu Thr Trp Gly
                                  (200)
AAG ATT TCA ACA GCA ATT CAT GAT GCC AAG AAT GGA GTT TTA CCC AAA CCT CTC
Lys Ile Ser Thr Ala lIe His Asp Ala Lys Asn Gly Val Leu Pro Lys Pro Leu
(210) (220)
GAG CTA GTG GAT GCC AGT GGT GCC AAG TGG ATA GTG TTG AGA GTG GAT GAA ATC
Glu Leu Val Asp Ala Ser Gly Ala Lys Trp Ile Val Leu Arg Val Asp Glu Ile
        (230) (240)
AAG CCT GAT GTA GCA CTC TTA AAC TAC GTT GCT GGG AGC TGT CAG ACA ACT TAT
Lys Pro Asp Val Ala Leu Leu Asn Tyr Val Gly Gly Ser Cys GIn Thr Thr Tyr
                (250) (260) (262)
AAC CAA AAT GCC ATG TTT CCT CAA CTT ATA ATG TCT ACT TAT TAT AAT TAC ATG GTT
Asn Gln Asn Ala Met Phe Pro Gln Leu Ile Met Ser Thr Tyr Tyr Asn Tyr Met Val
                        (270) (280)
                              (939)
AAT CTT GGT GAT CTA TTT GAA GGA TTC TGA TCA TAA ACA TAA TAA GGA GTA TAT ATA
Asn Leu Gly Asp Leu Phe Glu Gly Phe
                            (290)
TAT TAC TCC AAC TAT ATT ATA AAG CTT AAA TAA GAG GCC GTG TTA ATT AGT ACT TGT
TGC CTT TTG CTT TAT GGT GTT GTT TAT TAT GCC TTG TAT GCT TGT AAT ATT ATC TAG
AGA ACA AGA TGT ACT GTG TAA TAG TCT TGT TTG AAA TAA AAC TTC CAA TTA TGA TGC
AAA AAA AAA AAA AAA
```

### Example 3

### Binding of Native Eukaryotic Ribosomes and Synthetic Ribosomal Protein L3 to Mutant PAP Protein

Ribosome Binding Assays

Ribosomes were isolated from rabbit reticulocyte-rich whole blood (Pel-Freez Biologicals, Rogers, Ariz.) as described previously (Rajamohan, F., et al., 2001, Biochemistry, 40:9104-14). Total ribosomes (30 μg) were incubated with 5 μg of wild-type or mutant PAP proteins to a final volume of 100 μl in binding buffer and incubated at room temperature for 1 hour. After incubation, ribosomes were pelleted by centrifugation at 300,000×g for 30 minutes at 4° C. The pellets were washed two times with solution D (10 mM Tris-HCl, pH 7.5, 1 mM KCl, 0.1 M MgCl₂) and resuspended in 20 μl of PBS (137 mM NaCl, 2.7 mM KCl, 10 mM Na₂HP0₄, 1 mM KH₂PO₄, pH 7.4). The protein samples were resolved on a SDS-12% PAGE gel and transferred onto a polyvinylidene difluoride membrane (Bio-Rad) using the Bio-Rad Trans-Blot apparatus, as described previously (Rajamohan F, et al., Protein Expression and Purification, 16(2): 359-368; Rajamohan F, et al., 2000, Journal of Biological Chemistry, 275(5):3382-3390). The membrane was immunoblotted using a rabbit anti-PAP serum (1:2000 dilution) and horseradish peroxidase-conjugated goat anti-rabbit IgG (Sigma) as the primary and secondary antibodies, respectively.

L3 Binding Assays

A plasmid containing the cDNA (pJD166.trp) that encodes the wild-type Saccharomycis cerevisiae ribosomal protein L3 was a kind gift from Dr. Jonathan D. Dinman, University of Medicine and Dentistry of New Jersey. Radiolabeled L3 protein was synthesized by a linked transcription-translation system (TNT T3-coupled Reticulocyte Lysate System, Promega) (Rajamohan, F., et al., 2001, Biochemistry, 40:9104-14) according to the manufacturer's instructions (Promega). The translation products were resolved on a SDS-10% PAGE gel, which was dried and autoradiographed. The mouse anti-L3 monoclonal antibody was a kind gift from Dr. Jonathan R Warner, Department of Cell Biology, Albert Einstein College of Medicine, Bronx, N.Y. The in vitro synthesized L3 protein (8×10⁴ cpm) was incubated with 1 μg of wild-type or mutant PAP proteins in 50 μl (final volume) of binding buffer (10 mM K₂HPO₄, 5 mM NaCl, pH 8.0) at 30° C. for 30 minutes. The PAP-L3 complex was co-immunoprecipitated by adding 5 μl of the mouse anti-L3 monoclonal antibody (1:500 dilution) (Rajamohan, F., et al., 2001, Biochemistry, 40:9104-14). After 60 minutes of incubation at 30° C., the PAP-L3-antibody complex was precipitated by adding 50 μl of protein A-sepharose beads that had been pretreated with rabbit anti-mouse IgG (20 μl/ml beads) and continued the incubation for another 1 hour at 4° C. The beads were washed three times with phosphate-buffered saline containing 0.1% Triton X-100 and the proteins were eluted from the Sepharose beads with SDS sample buffer. The proteins were separated through SDS-12% PAGE, transferred to a PVDF membrane, and probed with the polyclonal rabbit anti-PAP antibody (1:2000 dilution) and horseradish peroxidase conjugated goat anti-rabbit IgG (1:1000 dilution) as the primary and secondary antibodies, respectively. The blot was developed using 3,3′-diaminobenzidine (Sigma) as the colorimetric indicator for peroxidase activity. The dried membrane was also exposed to autoradiography to estimate the amounts of L3 protein.

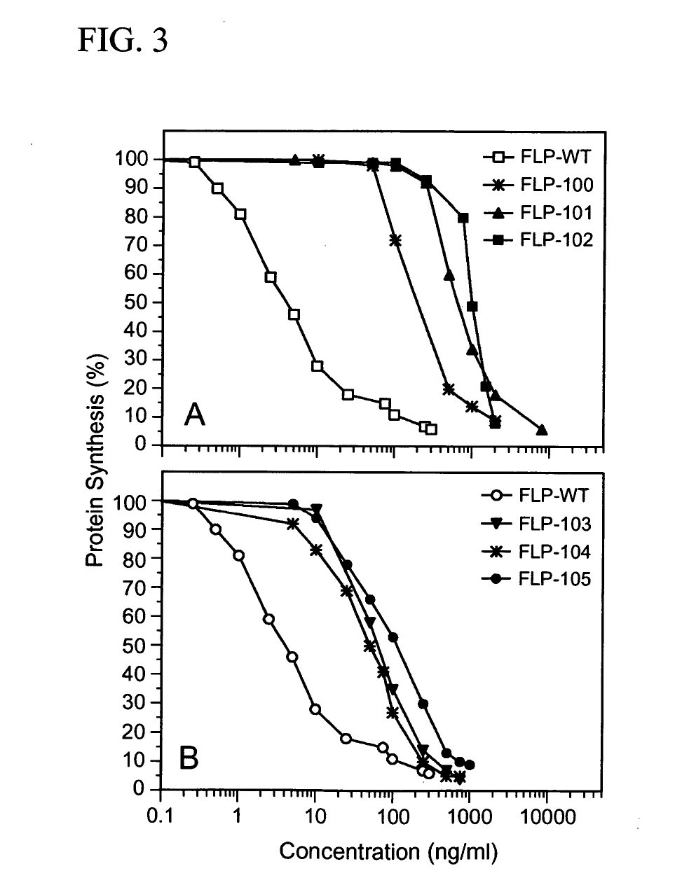

FIG. 3.

(A) Association of PAP mutants with ribosomes isolated from rabbit reticulocyte enriched blood. (A.1) Total ribosomal protein (5 μg) was incubated with 1 μg of PAP and the ribosome-PAP complexes were isolated by ultracentrifugation. The ribosome-PAP complexes were separated through SDS-12% PAGE, electroblotted onto a polyvinylidene difluoride (PVDF) membrane, and immunoblotted with a polyclonal antibody to PAP. (A.2) A fraction (5 μl) of the reaction mixture, prior to the separation of PAP-ribosome complex, was removed separated through SDS-12% PAGE, transferred to PVDF membrane, and immunoblotted with a polyclonal antibody to PAP. The results show that equal amounts of PAP proteins were added to the reaction mixture.

(B) Association of wild-type and mutant PAP proteins with in vitro synthesized ribosomal protein L3. (B.1) Co-immunoprecipitated PAP revealed by immunoblotting using anti-PAP antibody. ³⁵S-labeled L3 was incubated with wild-type and mutant PAP proteins and co-immunoprecipitated with protein A-Sepharose beads pre-coated with monoclonal antibody to L3. The PAP-L3 complexes were separated through SDS-12% PAGE, transferred to a PVDF membrane, and immunoblotted with a polyclonal anti-PAP antibody. (B.2) The blot was exposed to X-ray film, showing equal amounts of labeled L3 protein in each reaction. (B.3) A fraction (5 μl) of the reaction, prior to the co-immunoprecipitation, was removed from the reaction, separated through SDS-12% PAGE, transferred to a PVDF membrane, and immunoblotted with a polyclonal antibody to PAP. The results show that equal amounts of PAP were added to each reaction.

Results

In accord with the predictions of our modeling studies, the reduced ribosome inhibitory activity of FLP-102 or FLP-105 was not due to reduced binding to ribosomes (FIG. 3, panel A) or to the ribosomal protein L3 (FIG. 3, panel B), which serves as a docking site for PAP with the SR-loop.

### Example 4

### Effect of Mutant PAP Proteins on RNA Depurination of E. coli rRNA and HIV-1 RNA

E. coli Ribosomal RNA Depurination in Rabbit Ribosomes: Aniline Cleavage Assays

Five μg of E. coli 23S and 16S rRNA (Roche Molecular Biochemicals) with/or 30 μg of total ribosomes prepared from rabbit reticulocyte-rich whole blood (Rajamohan F, et al., 1999, BBRC, 260(2): 453-458) were incubated with increasing amounts of wild-type or mutant PAP protein in 50 μl (final volume) of binding buffer (25 mM Tris.HCl, pH 7.8, 10 mM KCl, 5 mM MgCl₂, 2% glycerol) at 37° C. for 1 hour. The rRNA was extracted with phenol:chloroform (24:24), precipitated with ethanol and treated with 20 μl of 1M aniline acetate (pH 4.5) for 30 minutes on ice. The rRNA was precipitated with ethanol, electrophoresed in a 6% urea/polyacrylamide gel, and stained with ethidium bromide as described previously (Rajamohan F, et al., 2000, Journal of Biological Chemistry, 275(5):3382-3390).

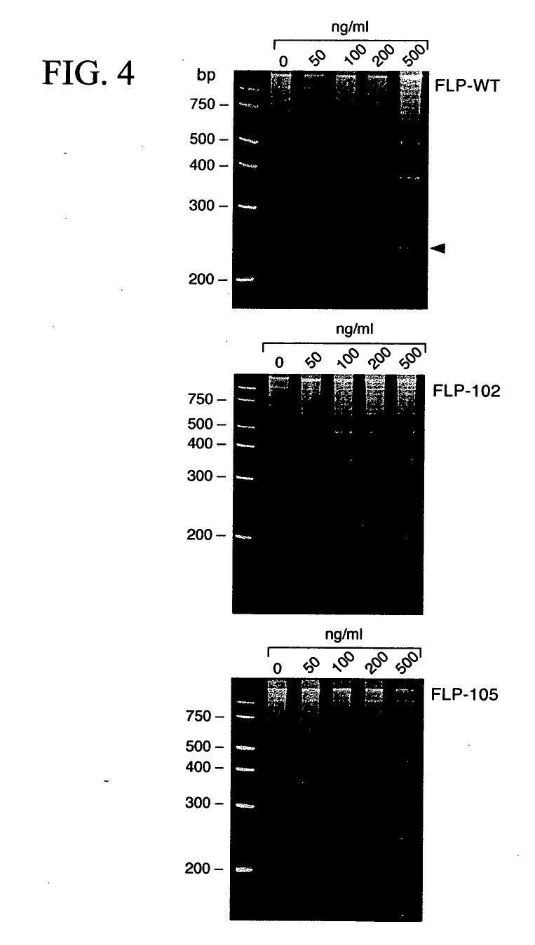

FIG. 4.

E. coli 23S+16S rRNA were treated with increasing amounts of PAP, treated with aniline, separated by 6% urea/polyacrylamide gel, and stained with ethidium bromide. The arrow indicates the fragment split by aniline.

Results: Aniline Cleavage of E. coli rRNA in Rabbit Ribosomes

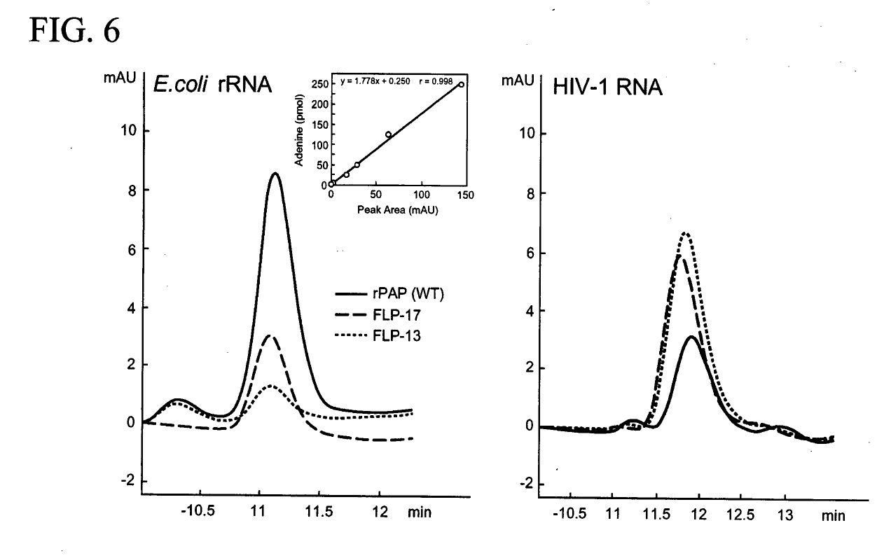

The ability of FLP-102 and FLP-105 proteins to depurinate E. coli rRNA in rabbit ribosomes was evaluated by treating the ribosomes with the PAP proteins, subsequent purification of E. coli rRNA, and cleavage with aniline. Aniline cleaves the sugar-phosphate backbone of rRNA at PAP depurination sites. Therefore, the release of fragments from aniline-treated rRNA is an indicator of PAP-mediated rRNA depurination. As shown in FIG. 4, aniline treatment resulted in the release of a 600 nt RNA fragment from E. coli rRNA in rabbit ribosomes pretreated with the recombinant wild-type PAP. In contrast, aniline treatment failed to cause the release of detectable amounts of the 600 nt RNA fragment from rRNA isolated from ribosomes treated with the mutant PAP proteins FLP-102 or FLP-105. These findings are consistent with the markedly reduced ribosome inhibitory activities of these recombinant PAP proteins in in vitro translation assays (FIG. 6, below). In accord with the predictions of our modeling studies, the reduced ribosome inhibitory activity of FLP-102 or FLP-105 was not due to reduced binding to ribosomes (FIG. 3 above, panel A) or the ribosomal protein L3 (FIG. 3 above, panel B) which serves as a docking site for PAP with the SR-loop.

HPLC-Based Quantitative Nucleotide-Release Assays of Ribosomal RNA Depurination

The release of adenine/guanine from E. coli 23S+16S rRNA (Roche Molecular Biochemicals) and HIV-1 RNA (ABI Biotechnologies, Columbia, Md.) was measured using an HPLC system (Hewlett Packard, Palo Alto, Calif.), equipped with a diode array detector and a ChemStation software program for data analysis, as described previously (Rajamohan F, et al., 1999, Biochemical and Biophysical Research Communications, 263(2): 419-424; Rajamohan F, et al., 1999, BBRC, 260(2): 453-458). Briefly, 2 μg of the RNA substrate was incubated with 2.5 μM of wild-type or mutant PAP proteins for 1 h at 37° C. in 50 μl of binding buffer (25 mM Tris-HCl, pH 7.8, 10 mM KCl, 5 MM MgCl₂, 2% glycerol). The reaction was stopped by adding 100 μl of HPLC running buffer (50 mM NH₄C₂H₃O₂, 5% methanol, pH 5.0) and 100 μl of the sample was injected automatically into a reverse-phase Lichrospher 100RP-18E analytical column (Hewlett-Packard, 5 mm particle size, 250×4 mm) that was equilibrated with HPLC running buffer as described previously (Rajamohan F, et al., 1999, Biochemical and Biophysical Research Communications, 263(2): 419-424; Rajamohan F, et al., 1999, BBRC, 260(2): 453-458). Controls included samples containing (a) untreated rRNA, and (b) test samples without rRNA. A calibration curve was generated to establish the linear relationship between the absolute peak area and the quantities of adenine/guanine (Sigma) as described previously (Rajamohan F, et al., 1999, Biochemical and Biophysical Research Communications, 263(2): 419-424; Rajamohan F, et al., 1999, BBRC, 260(2): 453-458). Unweighted linear regression analysis of the calibration curve was performed using the CA-Cricket graph III computer program (Computer Association, Inc., Islandia, N.Y.). Intra-assay and inter-assay accuracy and precisions were evaluated as described previously (Rajamohan, F., et al., 2001, Journal of Biological Chemistry, 276(26): 24075-81). Under the described chromatographic conditions, the retention times for adenine and guanine residues were 11.5 minutes and 5.7 minutes, respectively, and these were eluted without an interference peak from the blank controls. The lowest limit of detection of adenine was 2.5 pmol at a signal to noise ratio of approximately 3. The average peak area obtained for 50 and 250 pmol/50 μl of adenine standard was 29±1 and 143±7 mAU, respectively. The average peak area obtained for 50 and 250 pmol/50 μl of guanine standard was 31±2 and 156±8 mAU, respectively. The intra- and inter-assay coefficients of variation were less than 4%. The overall intra- and inter-assay accuracies of this method were 98.7±1.7% (N=5) and 95.7±3.0% (N=5), respectively.

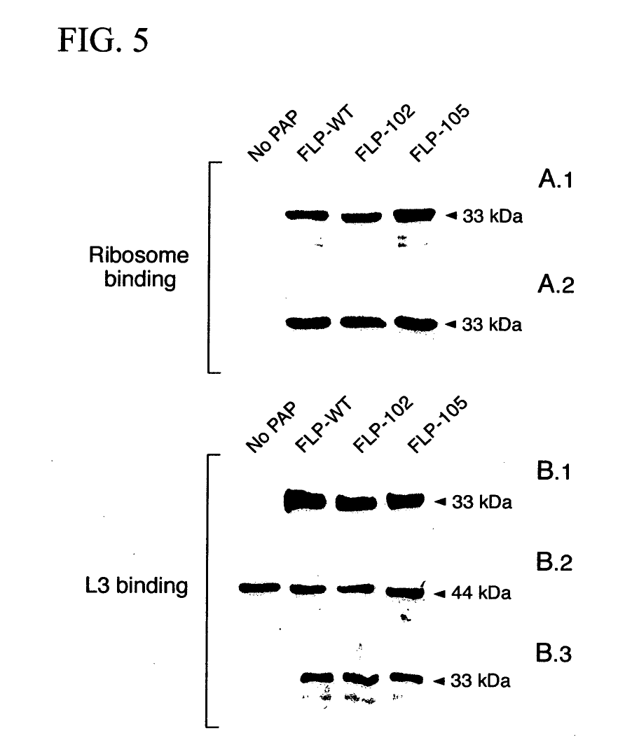

FIG. 5.

Two μg of E. coli rRNA or HIV-1 RNA were incubated with 2.5 μM of wild-type and mutant PAP proteins for 1 hour at 37° C. in 50 μl of binding buffer. The reaction was stopped by adding 100 μl of HPLC running buffer and 100 μl of the sample was injected in to the column, as described above. Control samples of RNA were treated with PBS instead of PAP. Inset: Standard curve of adenine and guanine standards.

TABLE 3
RNA Depurinating Activity and In Vivo Anti-HIV Activity of
Wild-type and Mutant PAP Proteins

| | Purine release | | | |
|---|---|---|---|---|
| | (pmol/μg RNA/ | | | Inhibition of |
| | μmol PAP protein) | | | HIV-1 Replication |
| | RNA | Adenine | Guanine | IC₅₀ (HTLV III₈) μg/mL |
| FLP-WT | E.coli | 76 ± 4 | 71 ± 3 | 2.9 ± 1.3 |
| | HIV-1 | 36 ± 3 | 48 ± 3 | |
| FLP-102 | E.coli | 7 ± 2 | 7 ± 2 | 0.2 ± 0.0 |
| | HIV-1 | 100 ± 7 | 207 ± 11 | |
| FLP-105 | E.coli | 17 ± 3 | 10 ± 4 | 0.7 ± 0.2 |
| | HIV-1 | 86 ± 7 | 180 ± 10 | |

Results: Adenine/Guanine Release Assays

The ability of the mutant PAP proteins FLP-102 and FLP-105 to depurinate ribosomal RNA versus HIV-1 RNA relative to that of recombinant wild-type PAP was compared using HPLC-based quantitative purine-release assays. While recombinant wild-type PAP depurinated ribosomal RNA more efficiently than HIV-1 RNA, both FLP-102 and FLP-105 were more efficient in depurinate HIV-1 RNA and their activity against HIV-1 RNA was superior to that of wild type PAP (Table 3). FLP-102 exhibited the most promising selective anti-HIV activity. The adenine release from E. coli ribosomal RNA for wild-type PAP protein was 76±4 pmols/μg RNA/μmols protein. FLP-102 protein was 10-fold less active and released only 7±2 pmols adenine/μg RNA/μmols protein (P<0.05, Table 3, FIG. 5). However, FLP-102 was more potent in deadenylating HIV-1 RNA than wild-type PAP. Wild-type PAP released 36±3 pmols adenine/μg RNA/μmols protein. FLP-102 released 100±7 pmols adenine/μg RNA/μmols protein (P<0.05) (Table 3, FIG. 5). Similar results were obtained in guanine-release assays (Table 3).

### Example 5

### Effect on Eukaryotic Protein Synthesis by Wild-Type and Mutant PAP Proteins

Cell-free Translation Assays

Protein synthesis was assayed in a cell-free system using nuclease-treated rabbit reticulocyte lysates (Promega, Madison, Wis.) and luciferase mRNA, as described previously (Rajamohan F, et al., Protein Expression and Purification, 16(2): 359-368; Rajamohan F, et al., 2000, Journal of Biological Chemistry, 275(5):3382-3390). Varying amounts of recombinant or mutant PAP proteins (0.1-100 ng/ml, pH solubilized) were added to the translation mixture containing: 10 μl of rabbit reticulocyte lysate, 0.5 μl of RNasin, 1.0 μl of 1 mM amino acid mixture (minus methionine), and 1.0 μl of [³⁵S]-methionine (10 mCi/ml, Amersham, Arlington Heights, Ill.). The final volume was adjusted to 19 μl with RNase-free water, followed by 15 minutes incubation at room temperature, and then 1.0 μl of 1:9 diluted luciferase mRNA (0.12 μg). After 2 hours, the incorporation of radioactive label into protein was determined by precipitating the synthesized luciferase protein (61 kDa) with 5% trichloroacetic acid according to the manufacturer's instructions (Promega). A minimum of ten concentrations of rPAP (WT) or mutant rPAP was used for the calculation of the IC₅₀ (50% inhibitory concentration) value. The adjusted IC₅₀ values (from precipitation assays) were then calculated by nonlinear regression analysis (Prism-2 Graph Pad software, San Diego, Calif.) using the average values of three independent experiments. The cpm values in control samples, with all the reagents added except the test sample, ranged from 3 to 4×10⁷ cpm/ml. These values were considered as 100% incorporation when determining the % control protein synthesis values for samples treated with test materials.

FIG. 6.

Each value is an average value obtained in three independent experiments. Protein synthesis was measured by [³⁵S]-methionine incorporation and the samples with all the reagents except PAP was assigned a value of 100% incorporation. (A) FLP-102 and single residue mutants FLP-100 and FLP-101. (B) FLP-105 and single residue mutants FLP-103 and FLP-104.

TABLE 4
Sequence Identity and Biological Activity of Wild-Type and
Mutant PAP Proteins

| | Original | | Translation* | IC₅₀ |
|---|---|---|---|---|
| | residue | Substituted | Inhibition IC₅₀ | IC₅₀ of |
| Mutants | numbered | residue | (ng/ml) | Wildtype rPAP |
| rPAP | | | 3.0 (2.5-3.8) | 1 |
| FLP-100 | K¹⁵¹ | A¹⁵¹ | 147 (130-167) | 49 |
| FLP-101 | I¹⁵² | A¹⁵² | 617 (500-710) | 206 |
| FLP-102 | ¹⁵¹KI¹⁵² | ¹⁵¹AA¹⁵² | 994 (878-1121) | 331 |
| FLP-103 | F¹⁹¹ | A¹⁹¹ | 57 (52-60) | 19 |
| FLP-104 | N¹⁹² | G¹⁹² | 54 (44-66) | 18 |
| FLP-105 | ¹⁹¹FN¹⁹² | ¹⁹¹AG¹⁹² | 105 (94-131) | 35 |

*95% Confidence limits are given in parentheses.
Each value is the mean of at least three experiments.

Results

The IC₅₀ value of recombinant wild-type PAP in cell-free eukaryotic translation inhibition assays was 3 ng/mL (FIG. 6, Table 4). By comparison, the IC₅₀ values for the double substitution mutants FLP-102 and FLP-105 were 994 ng/ml and 105 ng/ml, respectively. Thus, FLP-102 was 331-fold less toxic than wild type PAP and FLP-105 was 35-fold less toxic than wild type PAP (FIG. 6, Table 4). The corresponding single residue mutants FLP-100 (K151A), FLP-101 (I152A), FLP-103 (F191A) and FLP-104 (N192G) also were less toxic than the wild type PAP (FIG. 6, Table 4). As shown in Table 4, most of the 331 -fold activity loss for FLP-102 could be attributed to the I152A mutation since the single residue substitution mutant FLP-101 (I152A) was 206-fold less toxic but the single residue substitution mutant FLP-100 only 49-fold less toxic than wild-type PAP.

Compared to wild-type recombinant PAP protein, the described mutant PAP proteins (residue substitutions 151, 152, 191, 192, or combinations thereof) inhibition of eukaryotic mRNA translation (luciferase mRNA). Mutant PAP proteins with substitutions in residues 151 and/or 152 produced the lowest inhibition compared to wild-type PAP.

### Example 6

### Inhibition of Retroviral Replication (HIV-1) by Mutant PAP Protein

The anti-HIV-1 activities of wild-type and mutant rPAP (pH solubilized) proteins were evaluated by determining their ability to inhibit the replication of the HIV-1 strain, HTLV-IIIB, in normal human peripheral blood mononuclear cells (PBMC) from HIV-negative donors, as previously described (Uckun FM, et al., 1998, Antimicrob Agents Chemotherapy 42:383-388). The percentage of viral replication inhibition was calculated by comparing the mean p24 antigen/reverse transcriptase (RT) assay values for the test substance-treated infected cells with the p24/RT values for untreated infected cells (i.e., virus controls). The IC₅₀ values were calculated by nonlinear regression analysis (Prism-2 Graph Pad software) using the data from three independent experiments.

Results

Independent experiments, each performed in triplicate, examined the ability of FLP-102 and FLP-105 to inhibit replication of the HIV-1 strain, HTLVIIIB, in human peripheral blood mononuclear cells (PBMC) (Table 3). Both proteins inhibited the HIV-1 replication in a concentration dependent fashion with IC₅₀ values of 0.2±0.0 μg/mL for FLP-102 and 0.7±0.2 μg/mL for FLP-105. By comparison, the wild-type protein (FLP-WT, Table 3) was less active than either protein and inhibited the HIV-1 replication with an IC₅₀ value of 2.9±1.3 μg/mL (Table 3, P<0.001).

Results Summary:

Compared with the K151A and I152A mutations in FLP-102, the F191A and N192G mutations in FLP-105 had a lesser effect on PAP activity against the ribosomal and viral RNA substrates. Both N192G and F191A resulted in moderate reduction in ribosomal deactivation activity (depurination, Table 6, FIG. 6) and slightly improved activity against HIV RNA substrate (depurination, Table 6, FIG. 6). This is consistent with the observation that N192 and F191 are located in a more flexible environment than that of I152. In light of moderate changes in PAP activity as a result of these two mutations, the level of conformational change probably is less profound than that caused by I152A. Nevertheless, the F191A mutation probably adopts the same mechanism as proposed for the I152A mutation (leading to reduced activity), but with less impact.

### Example 7

### Toxicity Studies of Mutant PAP Protein in Balb/c Mice

BALB/c Mice.

All Balb/c mice used in this study were obtained from the specific pathogen-free (SPF) breeding facilities of Taconic (Germantown, N.Y.) at five weeks of age. All husbandary and experimental contacts made with the female Balb/c mice were performed in a controlled environment (12-hours light/12-hours dark photoperiod, 22±1° C., 60±10% relative humidity), which is fully accredited by the USDA (United States Department of Agriculture). All mice were housed in microisolator cages (Lab Products, Inc., Maywood, N.Y.) containing autoclaved bedding. The mice were allowed free access to autoclaved pellet food and tap water throughout the experiments. Animal studies were approved by Parker Hughes Institute Animal Care and Use Committee and all animal care procedures conformed to the Guide for the Care and Use of Laboratory Animals (National Research Council, National Academy Press, Washington D.C. 1996).

Toxicity Studies in Mice.

The toxicity profile of PAP proteins in Balb/c mice was examined using methods that have been previously reported for other experimental agents (Uckun F. M., et al., 1998, Clin. Cancer Res. 4, 1125-1134; Uckun F M, et al., 1998, Antimicrob Agents Chemotherapy 42:383-388). Female Balb/c mice were injected intravenously with bolus doses of native or recombinant PAP proteins in HEPES buffer at the 2.7, 4.1, 5.5, 6.8 and 8.2 mg/kg dose levels and were monitored daily for lethargy, cleanliness and morbidity. Control mice were treated with PAP-free HEPES buffer. At the time of death, necropsies were performed and the toxic effects of PAP proteins were assessed. For histopathologic studies, tissues were fixed in 10% neutral buffered formalin, dehydrated, and embedded in paraffin by routine methods. Glass slides with affixed 6 micron tissue sections were prepared and stained with Hemotoxylin and Eosin (H&E). No sedation or anesthesia was used throughout the experiments. Mice were monitored daily for mortality for determination of the day 30 LD₅₀ values. Animals were electively sacrificed on day 30 to determine the toxicity of STAMP by examining their blood chemistry profiles and blood counts as well as evaluating multiple organs for the presence of toxic lesions. Blood was collected by intracardiac puncture following anesthesia with ketamine:xylazine and immediately heparinized. The blood chemistry profiles were examined using a Synchron CX5CE Chemical Analyzer (Beckmnan Instruments, Inc., Fullerton, Calif.). Blood counts (red blood cells [RBC], white blood cells [WBC] and platelets [Plt]) were determined using a HESKA Vet ABC-Diff Hematology Analyzer (HESKA Corporation, Fort Collins, Colo.). Absolute neutrophil counts (ANC) and absolute lymphocyte counts (ALC) were calculated from WBC values after determining the percentages of neutrophils and lymphocytes by a manual differential count.

At the time of necropsy, 22 different tissues from mice (bone, bone marrow, brain, cecum, heart, kidney, large intestine, liver, lung, lymph node, ovary, pancreas, skeletal muscle, skin, small intestine, spleen, stomach, thymus, thyroid gland, urinary bladder, and uterus, as available) were collected within 15 minutes of sacrifice, gross pathological findings were documented, organs were preserved in 10% neutral phosphate buffered formalin, and processed for histopathological examination. For histopathologic studies, formalin fixed tissues were dehydrated and embedded in paraffin by routine methods. Glass slides with affixed 4-5 micron tissue sections were prepared and stained with Hemotoxylin and Eosin (H&E).

In Vivo Toxicity Profile and Anti-HIV Activity of FLP-102 and FLP-105.

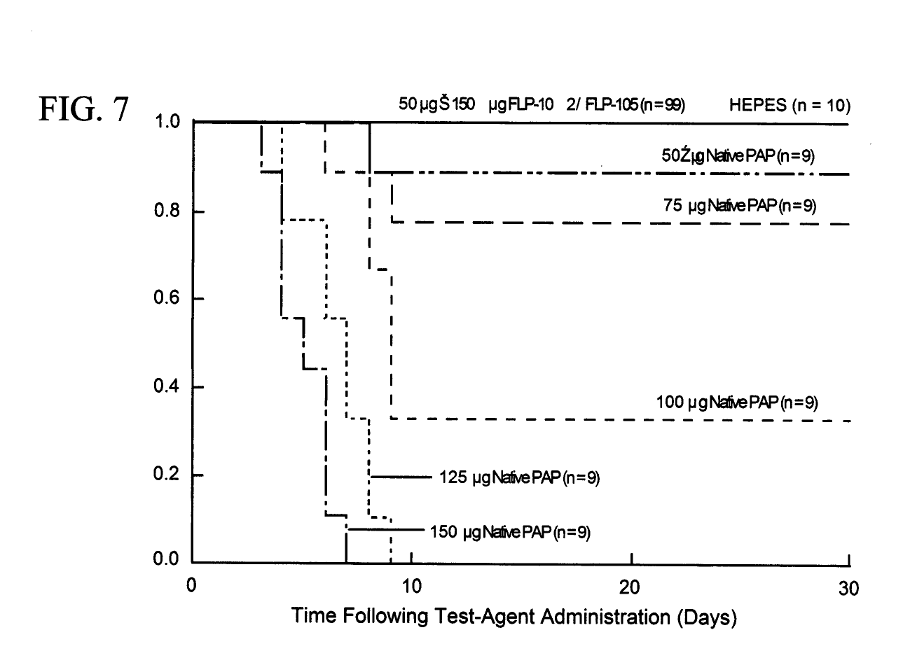

BALB/c mice were treated with 50 μg (n=10), 75 μg (n=10), 100 μg (n=10), 125 μg (n=10) or 150 μg (n=10) of FLP-102 or FLP-105. FLP-102 and FLP-105 were nontoxic to BALB/c mice even at a 150 μg/mouse (approximately 8.2 mg/kg, 18.2 g average weight) dose level, whereas wild type native PAP was exhibited toxicity at a 50 μg/mouse dose level and was invariably fatal at 125 μg/mouse or greater dose level (FIG. 7, Table 5). Control mice (n=10) were treated with i.p. injections of the PAP-free vehicle solution. All 49 FLP-102 -treated mice and all 50 FLP-105-treated mice remained healthy throughout the 30-day observation period with no evidence of morbidity. Blood tests done on day 30 did not suggest any significant systemic toxicity. In particular, even at the highest cumulative dose level of 150 μg/mouse, neither FLP-102 nor FLP-105 caused (a) anemia, neutropenia, or lymphopenia suggestive of hematologic toxicity, (b) elevations of BUN and creatinine or electrolyte disturbances suggestive of renal toxicity, (c) elevations of AST, ALT, alkaline phosphatase, LDH, or bilirubin suggestive of hepatotoxicity, or (d) elevation of amylase suggestive of pancreas toxicity (Table 5).

No toxic lesions were found in any of the 22 organs from the 25 (5/dose group) FLP-102 or 25 (5/dose group) FLP-105-treated mice sacrificed on day 30. Similarly, no toxic lesions were found in the organs of 10 vehicle-treated control mice. In contrast, myocardial necrosis, necrotizing hepatitis, necrotic myositis, and acute tubular necrosis were found when organs from native PAP (100-150 μg/mouse)-treated control mice were examined. Taken together, these experiments demonstrated that FLP-102 and FLP-105 are nontoxic to BALB/c mice at dose levels as high as 8.2 mg/kg.

FIG. 7.

BALB/c mice were treated with single i.p. bolus injections of either the mutant recombinant PAP proteins FLP-102 and FLP-105 or the native PAP protein at the indicated dose levels. Survival curves of mice treated at specific dose levels of the specified PAP proteins are depicted. None of the 99 mice treated with FLP-102 or FLP-105 at dose levels ranging from 50 μg/mouse to 150 μg/mouse became sick or died within the 30-day observation period.

TABLE 5
Effects of FLP-102 and FLP-105 on Health, Blood Chemistry Profiles, and Peripheral Blood Counts in Mice

| | | FLP102 | | | | |
|---|---|---|---|---|---|---|
| | Vehicle | 50 μg | 75 μg | 100 μg | 125 μg | 150 μg |
| | (n = 10) | (n = 10) | (n = 10) | (n = 10) | (n = 9) | (n = 10) |
| Morbidity | 0/10 | 0/10 | 0/10 | 0/10 | 0/9 | 0/10 |
| Weight Loss | 1/10 | 1/10 | 1/10 | 0/10 | 0/9 | 0/10 |
| Weight Gain | 9/30 | 9/10 | 9/10 | 10/10 | 9/9 | 10/10 |
| Mean Wt. Change | 2.0 ± 0.4 | 2.2 ± 0.4 | 1.9 ± 0.4 | 2.4 ± 0.2 | 3.0 ± 0.1 | 3.0 ± 0.3 |
| (g ± SEM [%]) | (11.2 ± 2.2) | (12.5 ± 2.6) | (11.1 ± 2.5) | (13.4 ± 1.1) | (13.2 ± 4.0) | (16.8 ± 2.0) |
| Bone Marrow Function | | | | | | |
| WBC (× 10⁹/L) | 4.6 ± 0.7 | 4.2 ± 0.3 | 6.2 ± 0.6 | 4.7 ± 0.5 | 5.0 ± 0.6 | 6.0 ± 0.8 |
| ANC (× 10⁹/L) | 1.4 ± 0.3 | 1.4 ± 0.3 | 1.6 ± 0.4 | 0.8 ± 0.1 | 0.8 ± 0.2 | 1.0 ± 0.2 |
| ALC (× 10⁹/L) | 3.1 ± 0.5 | 2.9 ± 0.2 | 4.3 ± 0.2 | 3.7 ± 0.4* | 4.1 ± 0.6 | 4.6 ± 0.8 |
| RBC (× 10¹²/L) | 8.8 ± 0.7 | 11.0 ± 0.5 | 10.3 ± 0.5 | 9.6 ± 0.5 | N.D. | 8.5 ± 0.8 |
| Renal Function/Metabolism | | | | | | |
| BUN (mg/dL) | 32.6 ± 1.4 | 33.2 ± 1.5 | 28.3 ± 1.3 | 27.3 ± 1.7 | 25.3 ± 0.7 | 24.5 ± 0.9 |
| Creatinine (mg/dL) | 0.6 ± 0.2 | 0.9 ± 0.1 | 0.7 ± 0.1 | 0.6 ± 0.1 | 0.6 ± 0.1 | 0.6 ± 0.1 |
| Albumin (g/dL) | 1.4 ± 0.0 | 1.4 ± 0.0 | 1.4 ± 0.0 | 1.4 ± 0.0 | 1.4 ± 0.0 | 1.4 ± 0.0 |
| Total Protein (g/dL) | 4.4 ± 0.1 | 4.6 ± 0.1 | 4.5 ± 0.1 | 4.5 ± 0.1 | 4.6 ± 0.1 | 4.5 ± 0.1 |
| Triglycerides (mg/dL) | 112.7 ± 7.6 | 118.2 ± 15.4 | 101.4 ± 6.1 | 145.4 ± 15.0 | 131.0 ± 26.3 | 151.3 ± 22.8 |
| Calcium (mg/dL) | 8.3 ± 0.1 | 8.7 ± 0.1 | 8.5 ± 0.1 | 8.5 ± 0.1 | 8.6 ± 0.1 | 8.6 ± 0.1 |
| Phosphate (mg/dL) | 10.3 ± 0.5 | 10.8 ± 0.4 | 10.3 ± 0.4 | 10.0 ± 0.4 | 9.8 ± 0.4 | 9.4 ± 0.4 |
| Liver Function | | | | | | |
| AST (IU/L) | 159.5 ± 19.7 | 168.5 ± 22.4 | 149.9 ± 13.0 | 114.1 ± 10.1 | 215.1 ± 52.0 | 247.5 ± 61.7 |
| LDL (IU/L) | 1929.4 ± 123.4 | 2091.9 ± 121.7 | 1514.9 ± 131.2 | 1081.2 ± 105.3 | 1417.7 ± 319.3 | 1270.3 ± 262.5 |
| Total Bilirubin (mg/dL) | 0.6 ± 0.1 | 0.6 ± 0.1 | 1.0 ± 0.2 | 0.9 ± 0.1 | 1.0 ± 0.1 | 0.6 ± 0.2 |
| Ammonia (mol/L) | 202.1 ± 20.8 | 131.6 ± 38.7 | 80.6 ± 15.1 | 40.0 ± 7.6 | 63.2 ± 20.4 | 99.2 ± 17.7 |
| Pancreas Function | | | | | | |
| Amylase (U/L) | 956.9 ± 31.6 | 1014.8 ± 60.2 | 987.7 ± 27.2 | 1036.6 ± 36.2 | 3050.7 ± 37.2 | 975.9 ± 26.7 |

| | FLP105 | | | | |
|---|---|---|---|---|---|
| | 50 μg | 75 μg | 100 μg | 125 μg | 150 μg |
| | (n = 10) | (n = 10) | (n = 10) | (n = 10) | (n = 10) |
| Morbidity | 1/10 | 0/10 | 0/10 | 0/10 | 0/10 |
| Weight Loss | 1/10 | 0/10 | 0/10 | 0/10 | 0/10 |
| Weight Gain | 9/10 | 10/10 | 10/10 | 10/10 | 10/10 |
| Mean Wt. Change | 4.8 ± 0.6 | 4.9 ± 0.3 | 5.6 ± 0.3 | 5.0 ± 0.4 | 4.8 ± 0.5 |
| (g ± SEM [%]) | (39.9 ± 5.8) | (37.3 ± 3.2) | (42.0 ± 2.8) | (39.1 ± 3.7) | (40.4 ± 6.2) |
| Bone Marrow Function | | | | | |
| WBC (× 10⁹/L) | 4.8 ± 0.7 | 4.1 ± 0.5 | 7.1 ± 1.0 | 6.8 ± 1.1 | 5.1 ± 0.5 |
| ANC (× 10⁹/L) | 1.3 ± 0.5 | 0.8 ± 0.2 | 1.1 ± 0.3 | 1.2 ± 0.2 | 1.5 ± 0.2 |
| ALC (× 10⁹/L) | 3.3 ± 0.4 | 3.2 ± 0.4 | 5.9 ± 0.9* | 5.4 ± 1.0 | 3.5 ± 0.4 |
| RBC (× 10¹²/L) | 8.4 ± 1.1 | 5.9 ± 1.1 | 8.3 ± 0.9 | 2.0 ± 0.8 | 7.6 ± 1.1 |
| Renal Function/Metabolism | | | | | |
| BUN (mg/dL) | 31.1 ± 1.2 | 28.4 ± 1.3 | 25.1 ± 0.7 | 26.3 ± 0.8 | 27.5 ± 0.7 |
| Creatinine (mg/dL) | 1.2 ± 0.2 | 0.8 ± 0.1 | 0.9 ± 0.1 | 0.6 ± 0.1 | 0.4 ± 0.1 |
| Albumin (g/dL) | 1.4 ± 0.0 | 1.4 ± 0.1 | 1.5 ± 0.1 | 1.4 ± 0.1 | 1.3 ± 0.0 |
| Total Protein (g/dL) | 4.1 ± 0.1 | 4.2 ± 0.1 | 4.5 ± 0.1 | 4.5 ± 0.1 | 4.4 ± 0.1 |
| Triglycerides (mg/dL) | 277.0 ± 21.6 | 230.0 ± 21.2 | 267.8 ± 36.2 | 238.0 ± 21.4 | 218.3 ± 34.4 |
| Calcium (mg/dL) | 7.9 ± 0.2 | 8.4 ± 0.1 | 8.4 ± 0.2 | 8.8 ± 0.4 | 8.3 ± 0.2 |
| Phosphate (mg/dL) | 11.7 ± 0.9 | 12.5 ± 1.3 | 9.3 ± 0.5 | 10.3 ± 0.6 | 11.5 ± 0.9 |
| Liver Function | | | | | |
| AST (IU/L) | 149.8 ± 32.9 | 104.7 ± 14.4 | 154.5 ± 43.9 | 272.2 ± 58.6 | 101.0 ± 15.1 |
| LDL (IU/L) | 1423.3 ± 160.3 | 1241.2 ± 232.1 | 636.2 ± 112.0 | 1491.7 ± 274.8 | 1103.1 ± 226.3 |
| Total Bilirubin (mg/dL) | 1.0 ± 0.1 | 0.9 ± 0.1 | 0.9 ± 0.2 | 1.2 ± 0.3 | 1.1 ± 0.2 |
| Ammonia (mol/L) | 868.8 ± 16.8 | 579.3 ± 74.6 | 914.7 ± 121.3 | 802.4 ± 150.4 | 711.7 ± 22.2 |
| Pancreas Function | | | | | |
| Amylase (U/L) | 1428.1 ± 136.3 | 1173.8 ± 91.8 | 2286.0 ± 663.4 | 1573.9 ± 284.2 | 1042.3 ± 6 |

### Example 8

### Antiviral Studies of Mutant PAP Protein in HIV-infected SCID Mice (Human AIDS Model)

SCID mouse model of human AIDS.

All CB-17 SCID mice used in the present study were purchased from Taconic Labs (Germantown, N.Y.) at 6-8 weeks of age and maintained in the Level 3 (BL-3) Containment Facility for Preclinical Research of the Parker Hughes Institute. All husbandry and experimental contact made with the mice maintained specific-pathogen-free conditions. The mice were housed in Micro-Isolator cages containing autoclaved food, water, and bedding. Trimethoprim-sulfamethoxazole (Bactrim) was added to the drinking water of the SCID mice three times a week. Human peripheral blood lymphocyte-SCID (Hu-PBL-SCID) mice were generated by reconstituting SCID mice by intraperitoneal injection of 10×10⁶ PBMC from seronegative volunteer donors, as previously reported (Bell J A, et al., 1992, Biochemistry, 31(14):3590-6). Two weeks after inoculation of the cells, mice were anesthetized with Isoflurane and then challenged by intraperitoneal injection of 1×10⁵ median tissue culture infectious doses (TCID₅₀) of cell-free BR/92/019, a genotypically and phenotypically NRTI-resistant HIV-1 isolate. SCID mice were infected with BR/92/019 in the BL-3 containment facility, and all manipulations were performed in a biosafety cabinet. PAP proteins were administered by intraperitoneal injections. Throughout the experimental period, mice were monitored daily for overall health and survival.

Two weeks after infection, Hu-PBL-SCID mice were electively killed, and their peritoneal lavage cells as well as spleen cells were examined for evidence of infection by an HIV-1 co-culture assay (Erice A, et al., 1993, Antimicrob Agents Chemother 37:835-838) and determination of the viral RNA load (copies/mL of peritoneal lavage fluid or copies/mg of spleen tissue) by RT PCR using the Organon Teknika's® Nuclisens™ HIV-1 QT assay kit. Extraction of RNA was done with silica (50 μl) utilizing standard Boom technology and the NucliSens™ Extractor. Standard amplification (NASBA—nucleic acid sequence-based amplification) and detection assay was performed according to the manufacturer's recommendations. Detection was based on electrochemiluminescent (ECL) labels that emit light due to chemical reactions occurring on the surface of an electrode. Differences in the proportional response rate in different drug treatment groups were analyzed using a Chi-Squared test of independence. To compare the HIV burden of SCID mice with PCR evidence of HIV infection, a logistic regression was fitted to obtain the dose level at which the mean HIV burden in the tested tissues was reduced by 50% (95% confidence intervals). For histopathologic studies, tissues were fixed in 10% neutral buffered formalin, dehydrated, and embedded in paraffin by routine methods. Glass slides with affixed 6-μm tissue sections were prepared, stained with hematoxylin-eosin, and submitted to the veterinary pathologist for examination in a blinded fashion.

TABLE 6
Anti-HIV Activity of FLP-102 and 105 Against BR/92/019
in Hu-PBL-SCID Mice

| | | | Log₁₀ [Viral Load] | | | | |
|---|---|---|---|---|---|---|---|
| | HIV-PCR⁺ | | in PCR⁺Tissues | | HIV-Culture⁺ | | |
| Treatment | Spleen | PL | Spleen | PL | SPL | PL | SPL ± PL |
| Control (vehicle) | 9/10 | 7/8 | 3.9 ± 0.3 | 4.3 ± 0.2 | 9/10 | N.D. | 9/10 |
| ZDV/3TC | 3/10 | 6/10 | 3.2 ± 0.3 | 4.0 ± 0.4 | N.D. | N.D. | N.D |
| FLP-102 | 2/10 | 0/9 | ˜2.9 (1.6,4.3) | N.A | 2/10 | N.D. | 3/10 |
| FLP-105 | 3/10 | 3/9 | 3.2 ± 0.3 | 4.2 ± 0.6 | N.D. | N.D. | 5/10 |

FLP-102 and FLP-105 were used at the daily dose level of 20 μg/mouse, 5 days/week×2 weeks, administered by i.p bolus injections. ZDV/3TC was used at a dose level of 8 mg ZDV+4 mg 3TC, administered via lavage twice daily for 5 days/week×2 weeks. All mice were reconstituted with 10×10⁶ PBMC and infected with 1×10⁵ TCID₅₀ of the HIV-1 isolate BR/92/019 2 weeks after reconstitution. Treatments were initiated immediately after the inoculation of the HIV-1 isolate.
Results

We next sought to determine if FLP-102 and FLP-105 exhibit any in vivo anti-HIV activity in the Hu-PBL-SCID mouse model of human AIDS. Control mice were treated either with vehicle alone (negative control treatment) or with ZDV+3TC (positive control treatment). FLP-102 and FLP-105 were used at the daily nontoxic dose levels of 20-40 μg/mouse (2-4 mg/kg), 5 days/week×2 weeks, administered by i.p bolus injections. ZDV/3TC combination was used at a dose level of 8 mg zidovudine +4 mg lamivudine/3TC, administered via gavage twice daily for 5 days/week×2 weeks. All mice were reconstituted with 10×10⁶ PBMC and infected with 1×10⁵ TCID₅₀ of the genotypically and phenotypically NRTI-resistant HIV-1 isolate BR/92/019 two weeks after reconstitution. Treatments were started immediately after the inoculation of the HIV-1 isolate. Spleen specimens from 9 of 10 (90%) vehicle-treated control mice were HIV-1 PCR-positive with an HIV RNA burden of 3.9±0.3 logs, whereas spleen specimens from only 3 of 10 (30%) ZDV/3TC-treated mice were HIV-1 PCR-positive with an HIV RNA burden of 3.2±0.3 logs (Table 6). By comparison, spleens from only 2 of 10 (20%) FLP-102 treated mice (average HIV RNA burden=2.9 logs) and 3 of 10 (30%) FLP-105-treated mice (average HIV RNA burden=3.2±0.3 logs) were HIV-1 PCR-positive. Thus, both FLP-102 and FLP-105 were at least as effective as the in preventing the in vivo HIV-1 replication in the spleen of Hu-PBL-SCID mice. Peritoneal lavage specimens from 7 of 8 vehicle-treated control mice and 6 of 10 ZDV/3TC-treated mice were HIV-1 PCR-positive. In contrast, none of peritoneal lavage specimens from 9 FLP-102 treated mice and the peritoneal lavage specimens from only 3 of the 9 FLP-105-treated mice showed PCR evidence of HIV-1 infection (Table 6). The in vivo anti-HIV activity of the mutant PAP proteins was further confirmed by HIV-11 cultures of spleen specimens. Whereas 9 of 10 spleen specimens and 9 of 10 spleen+lavage (mixed) specimens from vehicle-treated control mice were HIV-1 culture-positive, only 2 of 10 spleen specimens and 3 of 10 spleen+lavage specimens from FLP-102-treated mice were HIV-1 culture-positive. By comparison, 3 of 10 spleen specimens and 5 of 10 spleen+lavage specimens from FLP-105-treated mice were HIV-1 culture-positive. Thus both PAP mutants were at least as effective as the ZDV/3TC combination and FLP-102 was the most active PAP protein in preventing the HIV-1 replication in Hu-PBL-SCID mice.

The above specification, examples and data provide a complete description of the manufacture and use of the composition of the invention. Since many embodiments of the invention can be made without departing from the spirit and scope of the invention, the invention resides in the claims hereinafter appended.

1.-34. (canceled)

35. A modified pokeweed antiviral protein (MPAP) comprising substitution of one or more amino acids positioned at least 10 angstroms from Arg179 of wild-type PAP (SEQ ID NO:1), wherein said substitution of one or more amino acids modifies at least one hydrophobic contact between adjacent alpha-helicies.

36. The MPAP of claim 35, comprising substitution of one or more amino acids within α helix 4-loop-α helix 5 region.

37. The MPAP of claim 36, comprising substitution of one or more of the following amino acids: Tyr76, Lys151, Ile152, Phe158, Thr162, or Thr166.

38. The MPAP of claim 35, wherein amino acid Lys151 is substituted.

39. The MPAP of claim 35, comprising the substitution Lys151 Ala.

40. The MPAP of claim 35, wherein amino acid Ile152 is substituted.

41. The MPAP of claim 35, comprising the substitution Ile152Ala.

42. The MPAP of claim 35, comprising one or more substitution in a C-terminal portion of α helix 6 or within a helix adjacent to the C-terminal portion of α helix 6.

43. The MPAP of claim 42, comprising substitution of one or more of the following amino acids: Ile13, Tyr16, Ile142, Lys188, Phe191, or Asp192.

44. The MPAP of claim 35, comprising substitution of amino acid Phe191.

45. The MPAP of claim 35, comprising the substitution Phe191 Ala.

46. The MPAP of claim 35, comprising substitution of amino acid Asp192.

47. The MPAP of claim 35, comprising the substitution Asp192Gly.

48. The MPAP of claim 35, wherein the substitution comprises Lys151Ala, Ile152Ala, Phe191Ala, Asp192Gly, or combinations thereof.

49. The MPAP of claim 48, wherein the substitution comprises Lys151Ala and Ile152Ala.

50. The MPAP of claim 35, wherein the substitution comprises Phe191Ala and Asp192Gly.

51. A modified pokeweed antiviral protein (MPAP) comprising substitution of one or more amino acids positioned at least 10 angstroms distance from reference amino acid Arg179 of wild-type PAP (SEQ ID NO:1), wherein said substitution modifies hydrophobic packing between adjacent alpha-helicies, such that MPAP-mediated depurination of viral RNA is greater than MPAP-mediated depurination of ribosomal RNA.

52. The MPAP of claim 51, wherein the viral RNA comprises retroviral RNA.

53. The MPAP of claim 51, wherein the viral RNA comprises HIV-1 RNA.

54. The MPAP of claim 51, wherein the viral RNA comprises RNA of a drug resistant HIV-1 strain.

55. The MPAP of claim 51, wherein a ratio of MPAP-mediated depurination of viral RNA to PAP-mediated depurination of ribosomal RNA is greater than 5 to 1.

56. A modified pokeweed antiviral protein (MPAP) comprising substitution of one or more amino acids positioned at least 10 angstroms distance from Arg179 of wild-type PAP (SEQ ID NO:1), wherein said substitution modifies hydrophobic packing between adjacent alpha-helicies, such that MPAP-mediated depurination of viral RNA is increased relative to that mediated by wild-type PAP.

57. The MPAP of claim 56, wherein the viral RNA comprises retroviral RNA.

58. The MPAP of claim 57, wherein the viral RNA comprises HIV-1 RNA.

59. The MPAP of claim 57, wherein the viral RNA comprises RNA of a drug resistant HIV-1 strain.

60. The MPAP of claim 57, wherein a ratio of MPAP-mediated depurination of viral RNA to wild-type PAP-mediated depurination of viral RNA is greater than 2 to 1.

61. A composition comprising MPAP according to claim 35 and a pharmaceutically acceptable carrier.

62. The composition of claim 61, further comprising one or more nucleoside analog reverse transcriptase inhibitor (NRTI), non-nucleoside analog reverse transcriptase inhibitor (NNRTI), protease inhibitor (PI), or combinations thereof.

63. A method for inhibiting viral replication comprising contacting a virus with MPAP according to claim 35.

64. The method of claim 63, wherein the virus comprises human immunodeficiency virus (HIV).

65. The method of claim 63, wherein the virus comprises HIV-1 virus.

66. The method of claim 63, wherein the virus comprises a drug resistant HIV-1 virus.

67. The method of claim 63, wherein virus comprises a drug resistant HIV-1 virus resistant to one or more of the following drugs: nucleoside analog, non-nucleoside analog, or protease inhibitor.

68. A method for inducing depurination of viral RNA comprising contacting a virus with MPAP according to claim 35.

69. A method for treating viral infection in a subject in need thereof, comprising administering to the subject MPAP according to claim 35.

70. Use of MPAP according to claim 35 for inhibiting viral replication.

71. Use of MPAP according to claim 35 for inducing depurination of viral RNA.

72. Use of a MPAP according to claim 35 for manufacture of a medicament for treating viral infection.
